(19) United States
(12) Patent Application Publication
(10) Pub. No.: US 2026/0069222 A1
(43) Pub. Date: Mar. 12, 2026

(54) USER INTERFACES FOR MONITORING HEALTH

(71) Applicant: Apple Inc., Cupertino, CA (US)

(72) Inventor: Matthew W. CROWLEY, Sunnyvale, CA (US)

(21) Appl. No.: 19/390,331

(22) Filed: Nov. 14, 2025

Related U.S. Application Data
(63) Continuation of application No. 17/574,268, filed on Jan. 12, 2022.
(60) Provisional application No. 63/137,704, filed on Jan. 14, 2021.
(60) Provisional application No. 63/142,757, filed on Jan. 28, 2021.

(51) Int. Cl. A61B 5/00; A61B 5/02

(52) U.S. Cl. CPC A61B 5/746; A61B 5/02; A61B 5/6843; A61B 5/7275; A61B 5/7282; A61B 5/743

(57) ABSTRACT

The present disclosure generally relates to user interfaces for monitoring health. In some examples, an electronic device displays a user interface for tracking and/or monitoring received blood pressure measurement data over a selected time range. The user interface for tracking and/or monitoring the received blood pressure measurement data includes a measurement entry user-interactive graphical user interface object that initiates a process for the electronic device to receive blood pressure measurement data corresponding to a respective measurement indicator of the user interface that corresponds to a current day and the selected time range.

### CROSS-REFERENCE TO RELATED APPLICATIONS

This application is a continuation of U.S. application Ser. No. 17/574,268, entitled “USER INTERFACES FOR MONITORING HEALTH,” filed Jan. 12, 2022, which claims benefit to U.S. Provisional Application Ser. No. 63/137,704, filed Jan. 14, 2021, entitled “USER INTERFACES FOR MONITORING HEALTH,” and U.S. Provisional Application Ser. No. 63/142,757, filed Jan. 28, 2021, entitled “USER INTERFACES FOR MONITORING HEALTH,” the contents of each of which are hereby incorporated by reference in their entirety.

### FIELD

The present disclosure relates generally to computer user interfaces, and more specifically to techniques for managing and/or presenting health data.

### BACKGROUND

Electronic devices include user interfaces that display various health information to a user. The health information can include data collected by sensors of the electronic devices and/or data input by the user.

### BRIEF SUMMARY

Some techniques for managing and/or presenting health data using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for managing and/or presenting health data. Such methods and interfaces optionally complement or replace other methods for managing and/or presenting health data. Such methods and interfaces reduce the cognitive burden on a user and produce a more efficient human-machine interface. For battery-operated computing devices, such methods and interfaces conserve power and increase the time between battery charges. Such methods and interfaces also provide a user with notifications related to health events, thereby providing the user with information related to his or her health without requiring input. Such methods and interfaces also enable a user to conveniently track health information, thereby facilitating a user's ability monitor his or her health.

In accordance with some embodiments, a method performed at a computer system that is in communication with one or more output devices and one or more sensors is described. The method comprises: receiving first data associated with a user of the computer system from the one or more sensors, where the first data from the one or more sensors does not include a direct measurement of pressure; and in response to receiving the first data from the one or more sensors: in accordance with a determination that the first data from the one or more sensors satisfies a set of hypertension notification criteria, generating, via the one or more output devices, a notification indicative of a hypertension event associated with the user of the computer system; and in accordance with a determination that the first data from the one or more sensors does not satisfy the set of hypertension notification criteria, forgoing generating the notification of the hypertension event.

In accordance with some embodiments, a non-transitory computer-readable storage medium storing one or more programs configured to be executed by one or more processors of a computer system that is in communication with one or more output devices and one or more sensors is described. The one or more programs include instructions for: receiving first data associated with a user of the computer system from the one or more sensors, where the first data from the one or more sensors does not include a direct measurement of pressure; and in response to receiving the first data from the one or more sensors: in accordance with a determination that the first data from the one or more sensors satisfies a set of hypertension notification criteria, generating, via the one or more output devices, a notification indicative of a hypertension event associated with the user of the computer system; and in accordance with a determination that the first data from the one or more sensors does not satisfy the set of hypertension notification criteria, forgoing generating the notification of the hypertension event.

In accordance with some embodiments, a transitory computer-readable storage medium storing one or more programs configured to be executed by one or more processors of a computer system that is in communication with one or more output devices and one or more sensors is described. The one or more programs include instructions for: receiving first data associated with a user of the computer system from the one or more sensors, where the first data from the one or more sensors does not include a direct measurement of pressure; and in response to receiving the first data from the one or more sensors: in accordance with a determination that the first data from the one or more sensors satisfies a set of hypertension notification criteria, generating, via the one or more output devices, a notification indicative of a hypertension event associated with the user of the computer system; and in accordance with a determination that the first data from the one or more sensors does not satisfy the set of hypertension notification criteria, forgoing generating the notification of the hypertension event.

In accordance with some embodiments, a computer system comprising one or more output devices, one or more sensors, one or more processors, and memory storing one or more programs configured to be executed by the one or more processors is described. The one or more programs including instructions for: receiving first data associated with a user of the computer system from the one or more sensors, where the first data from the one or more sensors does not include a direct measurement of pressure; and in response to receiving the first data from the one or more sensors: in accordance with a determination that the first data from the one or more sensors satisfies a set of hypertension notification criteria, generating, via the one or more output devices, a notification indicative of a hypertension event associated with the user of the computer system; and in accordance with a determination that the first data from the one or more sensors does not satisfy the set of hypertension notification criteria, forgoing generating the notification of the hypertension event.

In accordance with some embodiments, a computer system is described. The computer system comprises: one or more output devices; one or more sensors; and means for receiving first data associated with a user of the computer system from the one or more sensors, where the first data from the one or more sensors does not include a direct measurement of pressure; and means for, in response to receiving the first data from the one or more sensors: in accordance with a determination that the first data from the one or more sensors satisfies a set of hypertension notification criteria, generating, via the one or more output devices, a notification indicative of a hypertension event associated with the user of the computer system; and in accordance with a determination that the first data from the one or more sensors does not satisfy the set of hypertension notification criteria, forgoing generating the notification of the hypertension event.

In accordance with some embodiments, a method performed at a computer system that is in communication with a display generation component and one or more input devices is described. The method comprises: receiving, via the one or more input devices, a first set of one or more inputs that includes a first input selecting a time range; in response to receiving the first set of one or more inputs, displaying, via the display generation component, a first user interface, where the first user interface includes: in accordance with a determination that the first user input corresponds to selection of a first time range, a first plurality of measurement indicators within the first time range; in accordance with a determination that the first user input corresponds to selection of a second time range, different from the first time range, a second plurality of measurement indicators within the second time range; and a first measurement entry user-interactive graphical user interface object that, when selected via the one or more input devices, initiates a measurement-entry process for: in accordance with the first user interface including the first plurality of measurement indicators within the first time range, entering a blood pressure measurement that corresponds to the first time range and a current day; and in accordance with the first user interface including the second plurality of measurement indicators within the second time range, entering a blood pressure measurement that corresponds to the second time range and the current day.

In accordance with some embodiments, a non-transitory computer-readable storage medium storing one or more programs configured to be executed by one or more processors of a computer system that is in communication with a display generation component and one or more input devices is described. The one or more programs include instructions for: receiving, via the one or more input devices, a first set of one or more inputs that includes a first input selecting a time range; in response to receiving the first set of one or more inputs, displaying, via the display generation component, a first user interface, where the first user interface includes: in accordance with a determination that the first user input corresponds to selection of a first time range, a first plurality of measurement indicators within the first time range; in accordance with a determination that the first user input corresponds to selection of a second time range, different from the first time range, a second plurality of measurement indicators within the second time range; and a first measurement entry user-interactive graphical user interface object that, when selected via the one or more input devices, initiates a measurement-entry process for: in accordance with the first user interface including the first plurality of measurement indicators within the first time range, entering a blood pressure measurement that corresponds to the first time range and a current day; and in accordance with the first user interface including the second plurality of measurement indicators within the second time range, entering a blood pressure measurement that corresponds to the second time range and the current day.

In accordance with some embodiments, a transitory computer-readable storage medium storing one or more programs configured to be executed by one or more processors of a computer system that is in communication with a display generation component and one or more input devices is described. The one or more programs include instructions for: receiving, via the one or more input devices, a first set of one or more inputs that includes a first input selecting a time range; in response to receiving the first set of one or more inputs, displaying, via the display generation component, a first user interface, where the first user interface includes: in accordance with a determination that the first user input corresponds to selection of a first time range, a first plurality of measurement indicators within the first time range; in accordance with a determination that the first user input corresponds to selection of a second time range, different from the first time range, a second plurality of measurement indicators within the second time range; and a first measurement entry user-interactive graphical user interface object that, when selected via the one or more input devices, initiates a measurement-entry process for: in accordance with the first user interface including the first plurality of measurement indicators within the first time range, entering a blood pressure measurement that corresponds to the first time range and a current day; and in accordance with the first user interface including the second plurality of measurement indicators within the second time range, entering a blood pressure measurement that corresponds to the second time range and the current day.

In accordance with some embodiments, a computer system comprising a display generation component, one or more input devices, one or more processors, and memory storing one or more programs configured to be executed by the one or more processors is described. The one or more programs including instructions for: receiving, via the one or more input devices, a first set of one or more inputs that includes a first input selecting a time range; in response to receiving the first set of one or more inputs, displaying, via the display generation component, a first user interface, where the first user interface includes: in accordance with a determination that the first user input corresponds to selection of a first time range, a first plurality of measurement indicators within the first time range; in accordance with a determination that the first user input corresponds to selection of a second time range, different from the first time range, a second plurality of measurement indicators within the second time range; and a first measurement entry user-interactive graphical user interface object that, when selected via the one or more input devices, initiates a measurement-entry process for: in accordance with the first user interface including the first plurality of measurement indicators within the first time range, entering a blood pressure measurement that corresponds to the first time range and a current day; and in accordance with the first user interface including the second plurality of measurement indicators within the second time range, entering a blood pressure measurement that corresponds to the second time range and the current day.

In accordance with some embodiments, a computer system is described. The computer system comprises: a display generation component; one or more input devices; and means for receiving, via the one or more input devices, a first set of one or more inputs that includes a first input selecting a time range; means for, in response to receiving the first set of one or more inputs, displaying, via the display generation component, a first user interface, where the first user interface includes: in accordance with a determination that the first user input corresponds to selection of a first time range, a first plurality of measurement indicators within the first time range; in accordance with a determination that the first user input corresponds to selection of a second time range, different from the first time range, a second plurality of measurement indicators within the second time range; and a first measurement entry user-interactive graphical user interface object that, when selected via the one or more input devices, initiates a measurement-entry process for: in accordance with the first user interface including the first plurality of measurement indicators within the first time range, entering a blood pressure measurement that corresponds to the first time range and a current day; and in accordance with the first user interface including the second plurality of measurement indicators within the second time range, entering a blood pressure measurement that corresponds to the second time range and the current day.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing and/or presenting health data, thereby increasing the effectiveness, efficiency, and user satisfaction with such devices. Such methods and interfaces may complement or replace other methods for managing and/or presenting health data.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6P illustrate exemplary user interfaces for generating a notification associated with a health event in accordance with some embodiments.

FIG. 7 is a flow diagram illustrating a method for generating a notification associated with a health event in accordance with some embodiments.

FIGS. 8A-8U illustrate exemplary user interfaces for managing health information over a predefined time range in accordance with some embodiments.

FIG. 9 is a flow diagram illustrating a method for managing health information over a predefined time range in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for managing and/or presenting health data. For example, there is a need for electronic devices that monitor health data and notify a user of a potential health event without requiring periodic and/or intermittent user input over time. For another example, there is a need for electronic devices that facilitate a user's ability to log and/or track health information over time, such that a user can easily monitor his or her health. Such techniques can reduce the cognitive burden on a user who accesses health data on an electronic device, thereby enhancing productivity. Further, such techniques can reduce processor and battery power otherwise wasted on redundant user inputs.

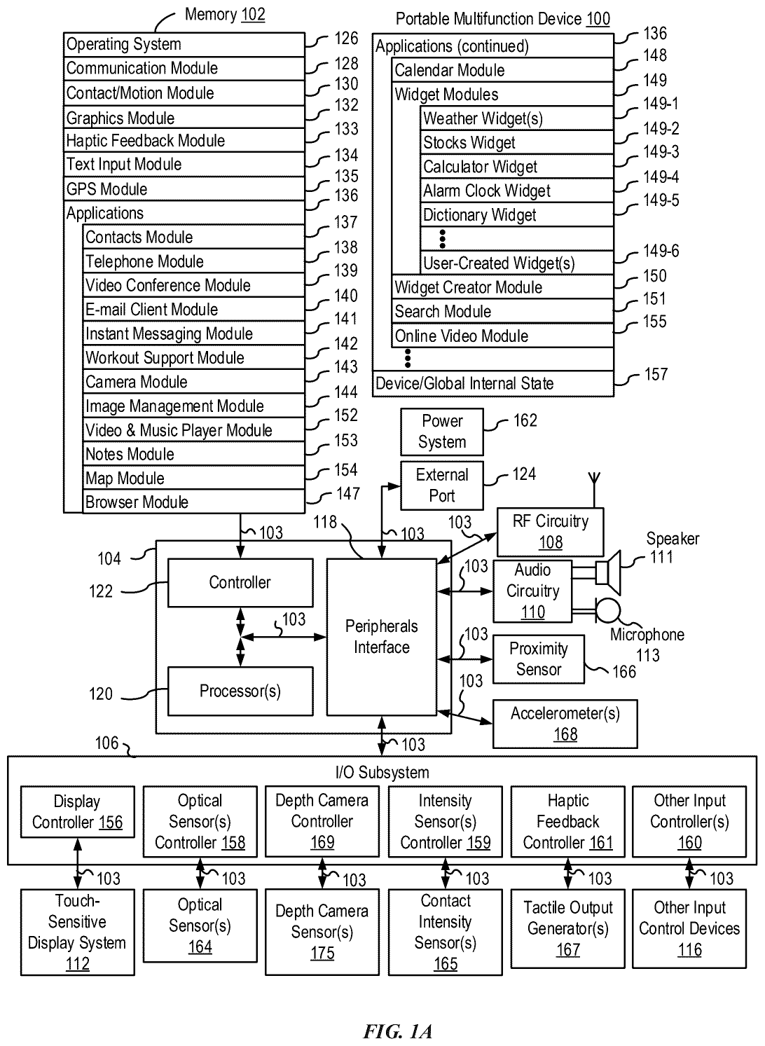

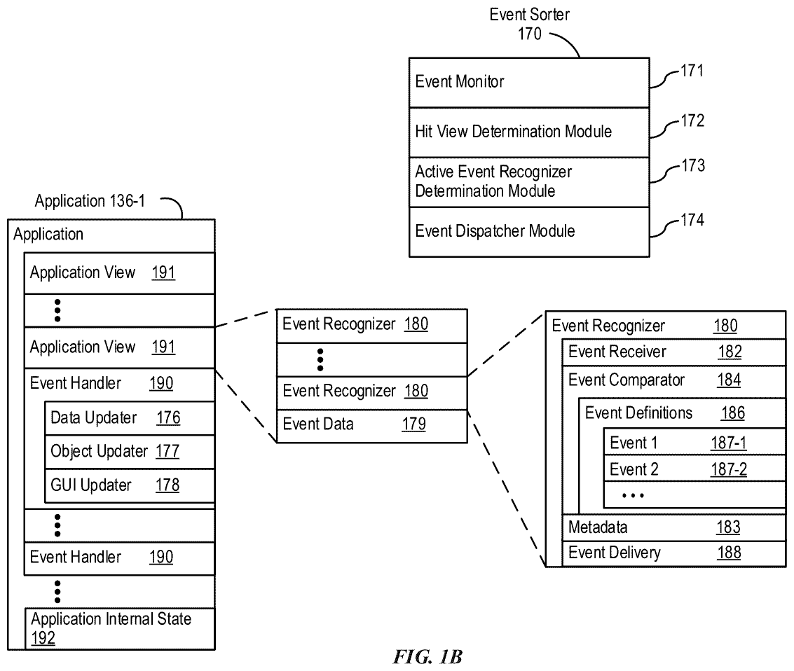

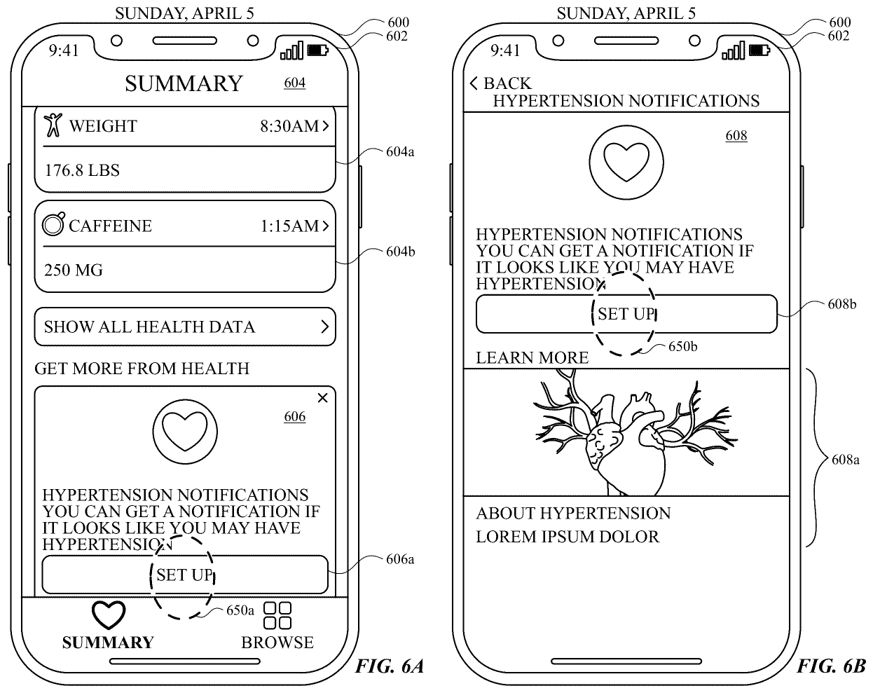

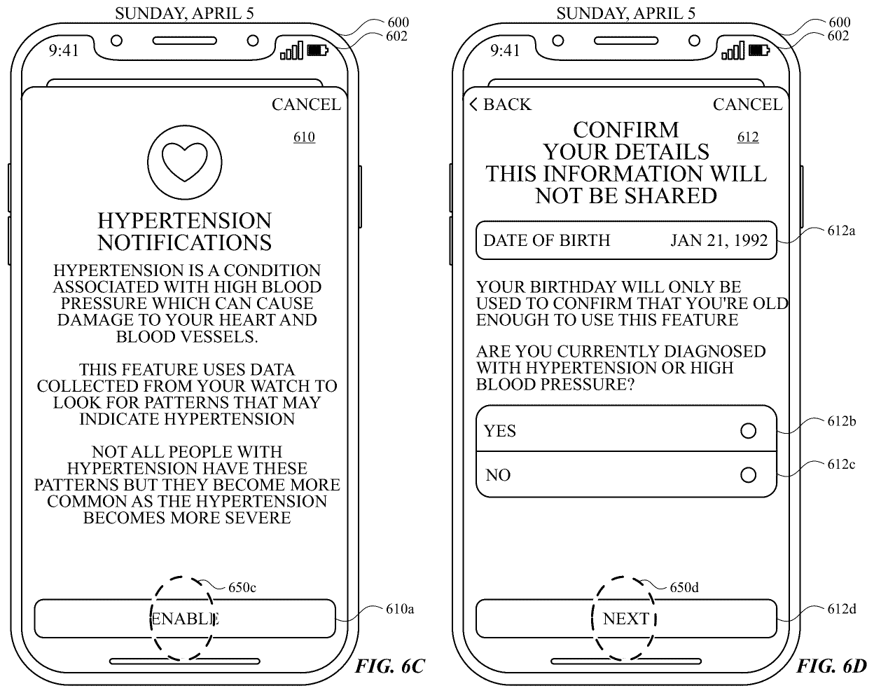

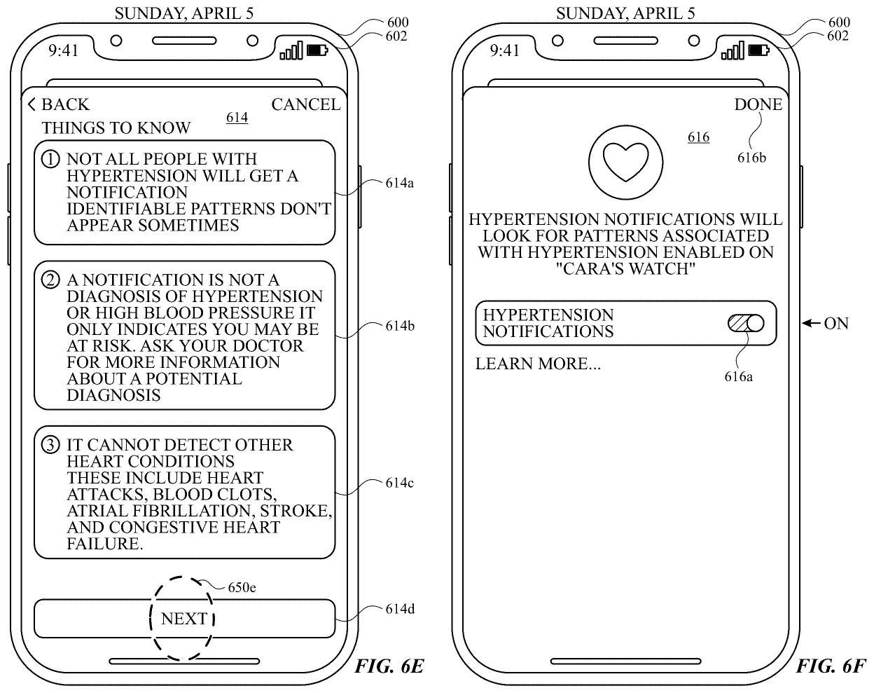

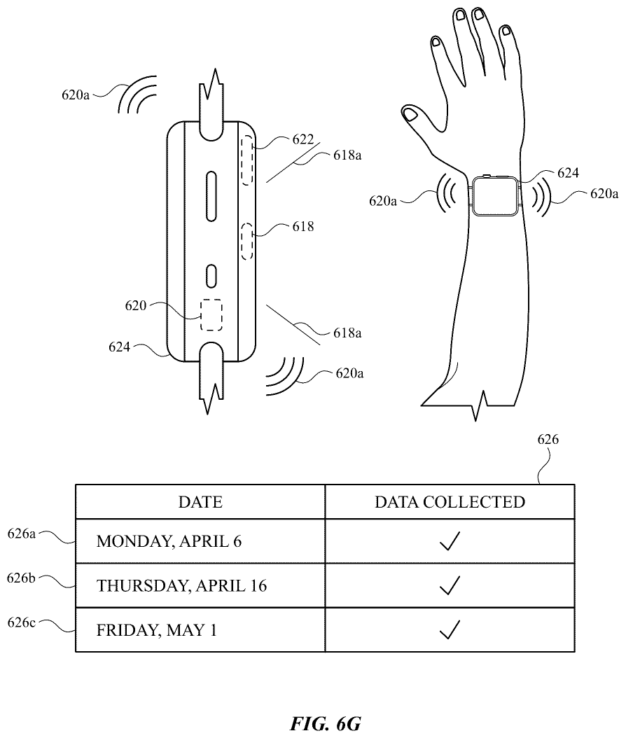

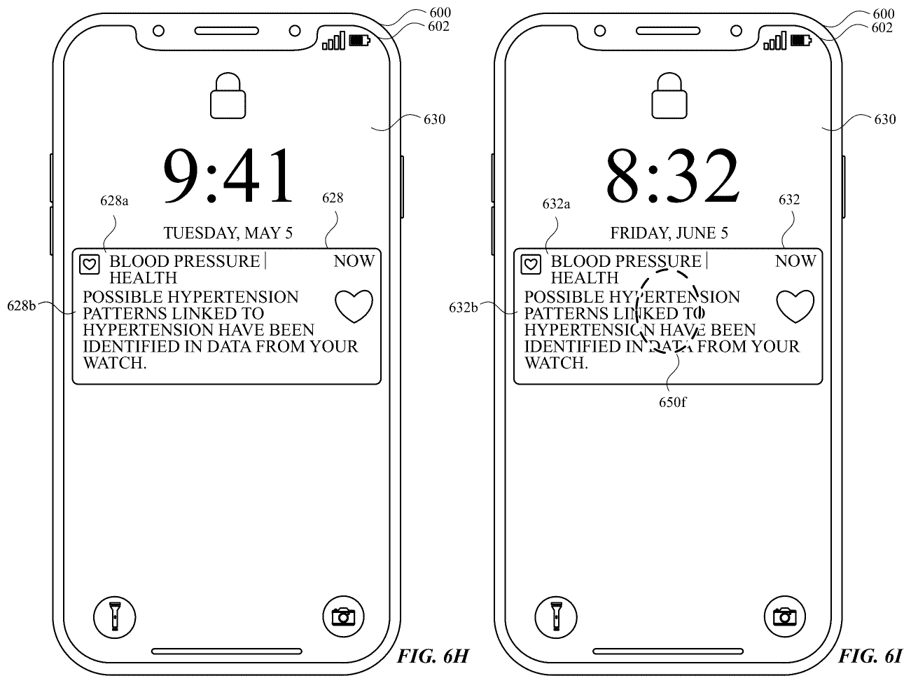

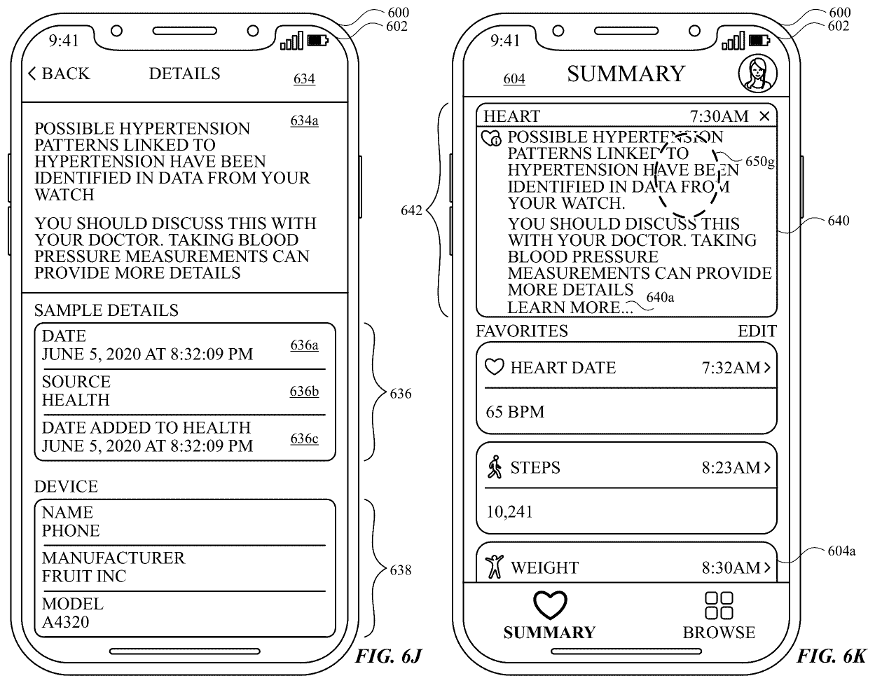

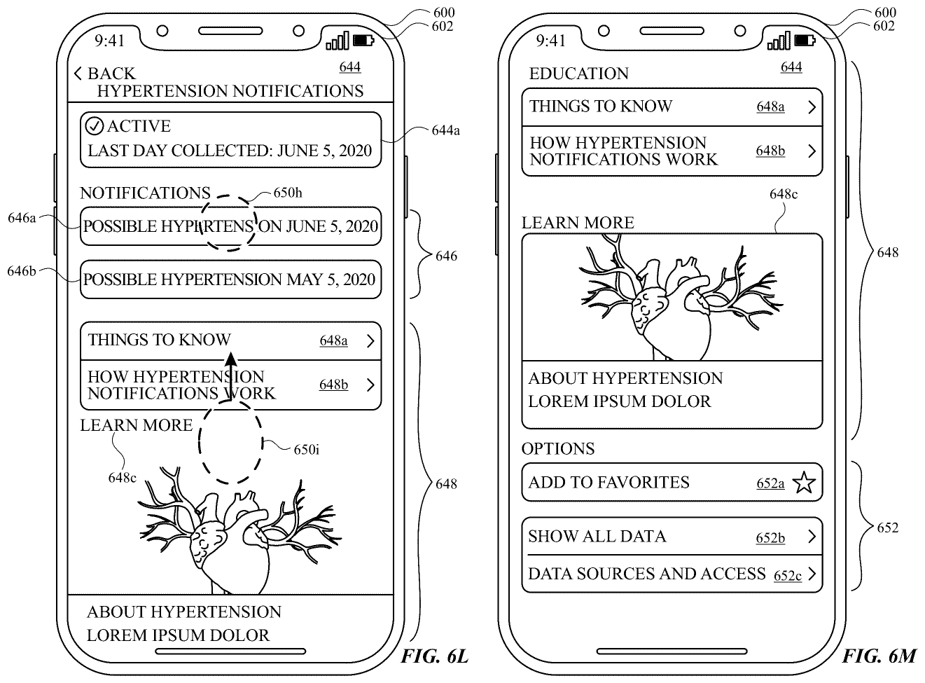

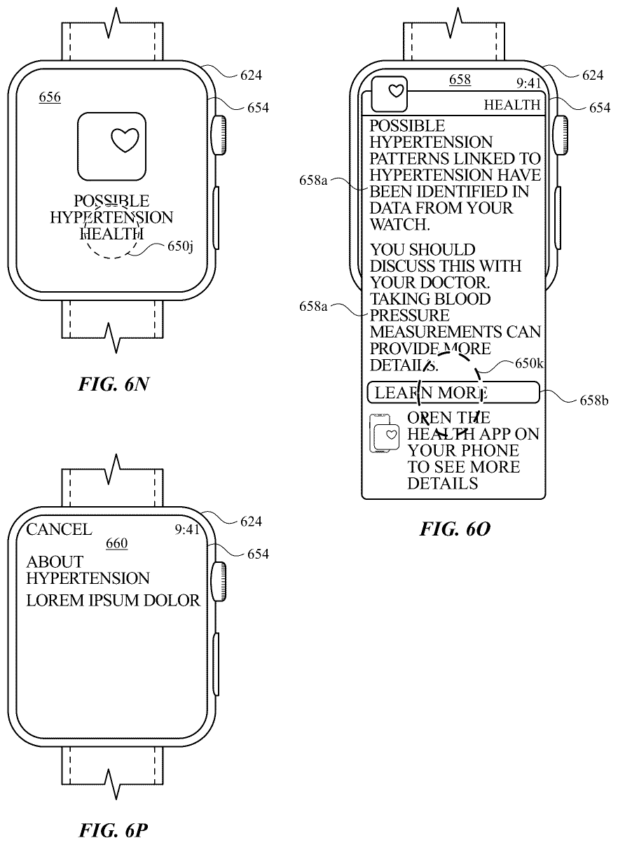

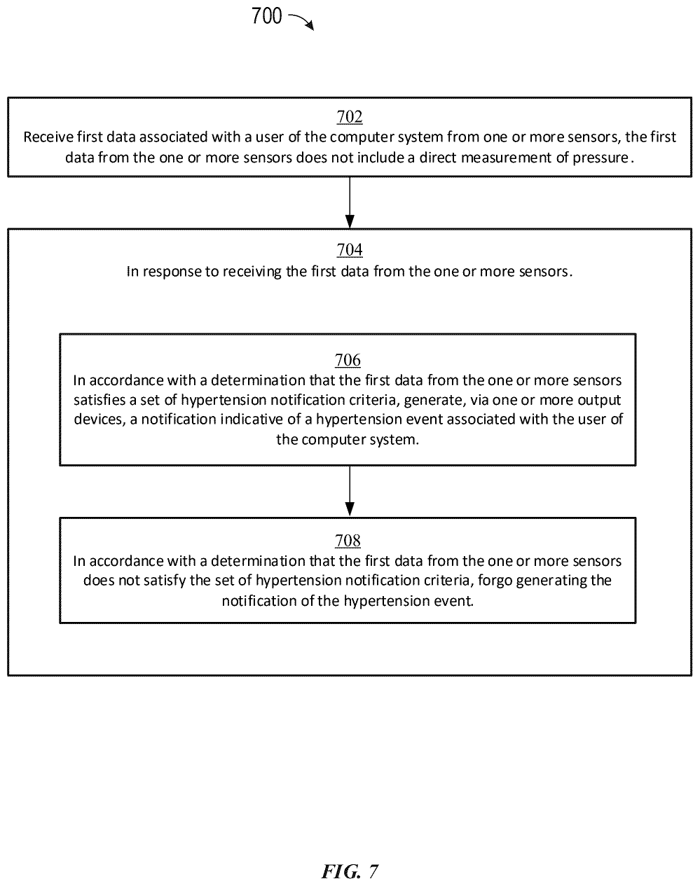

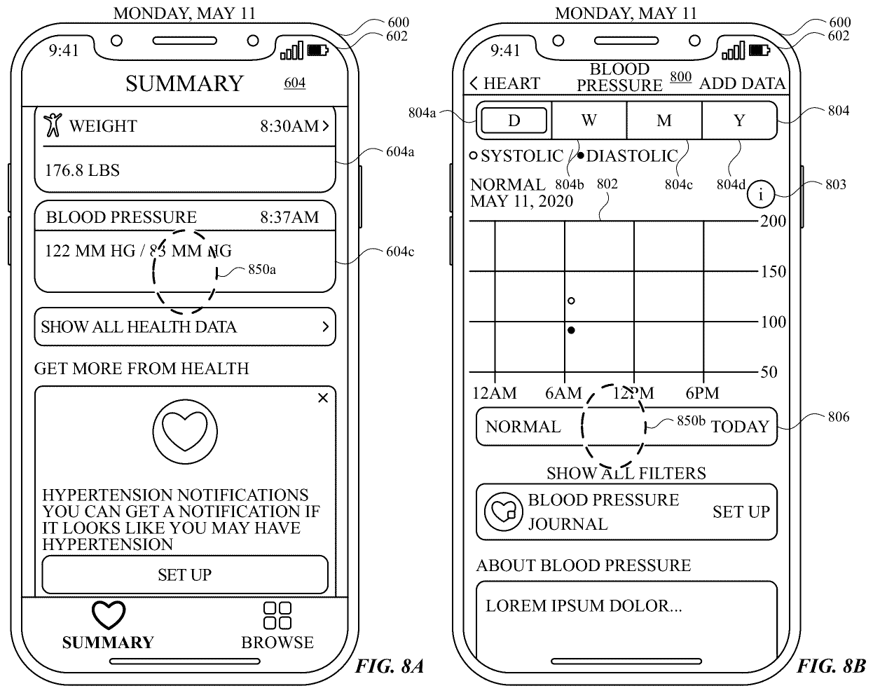

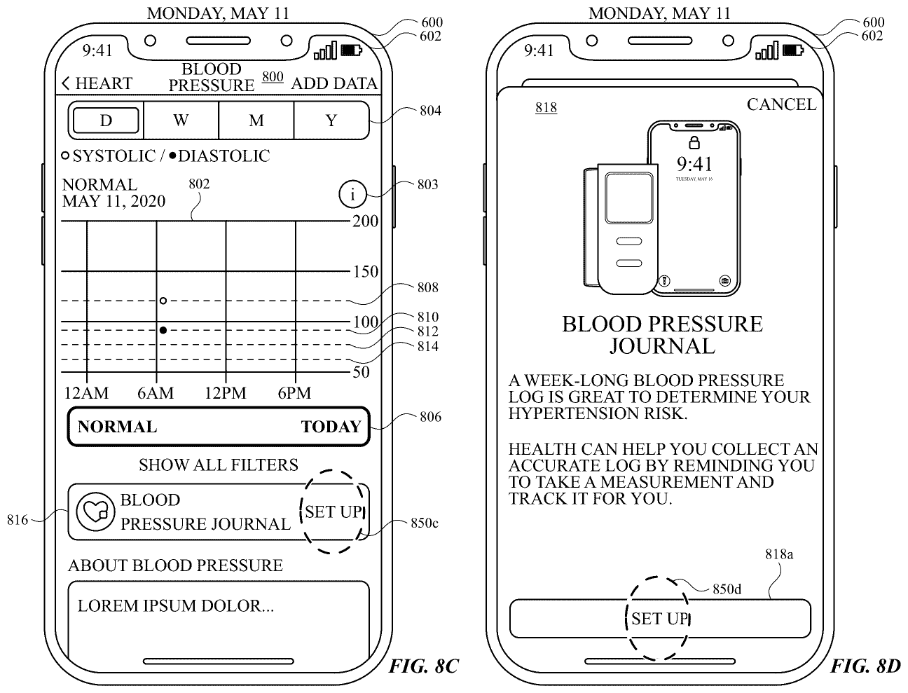

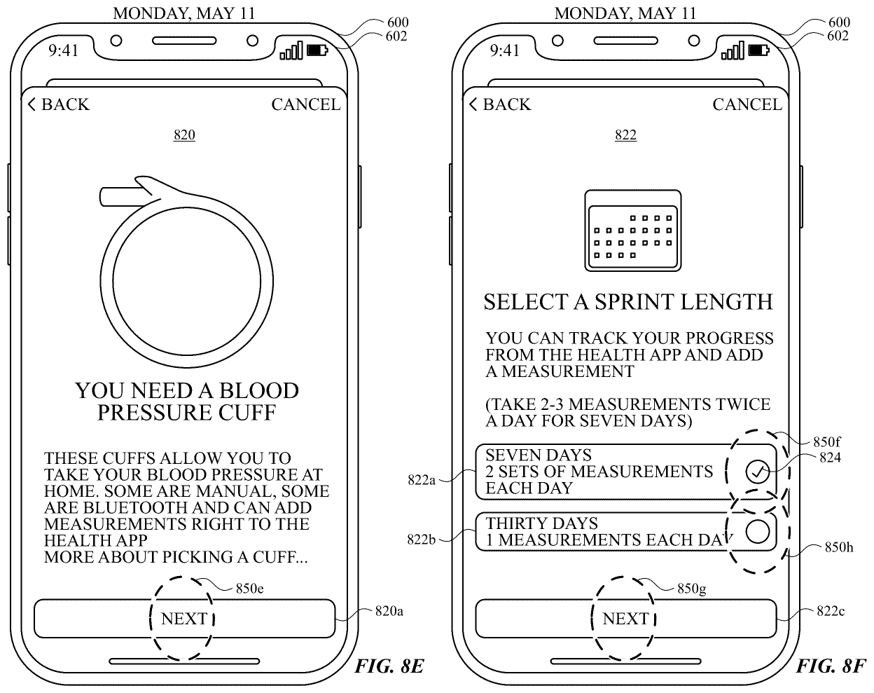

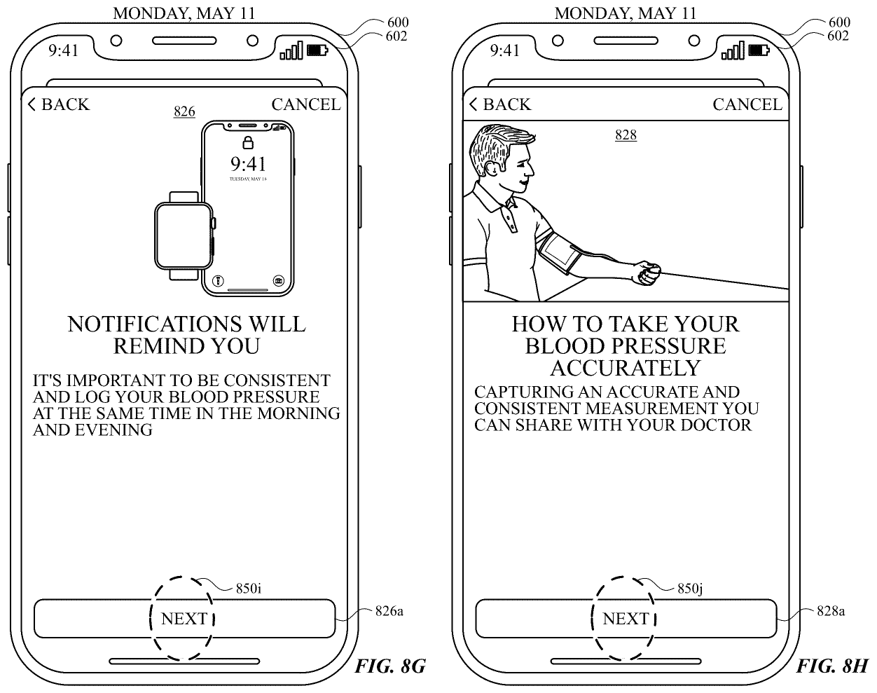

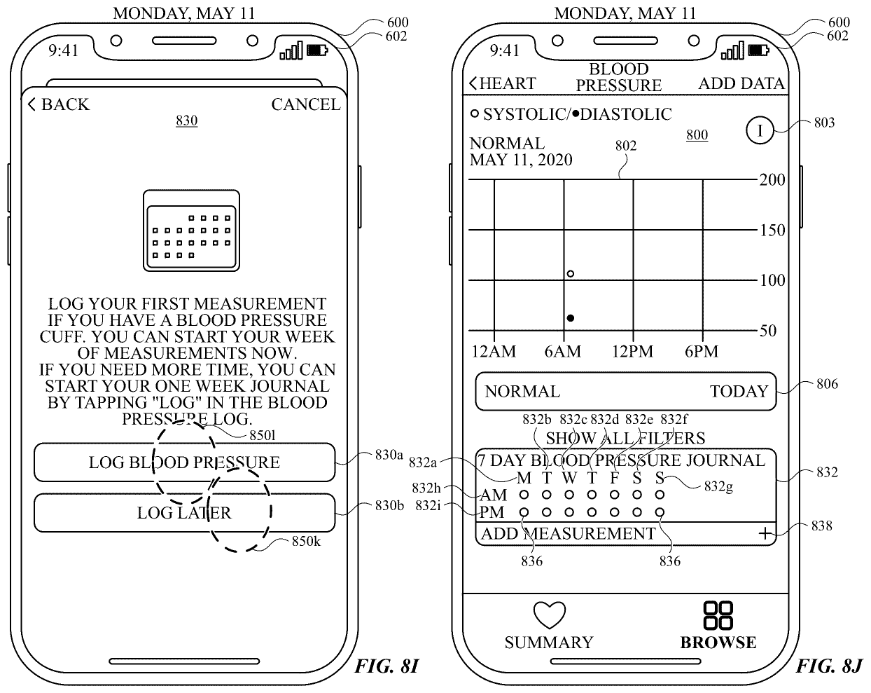

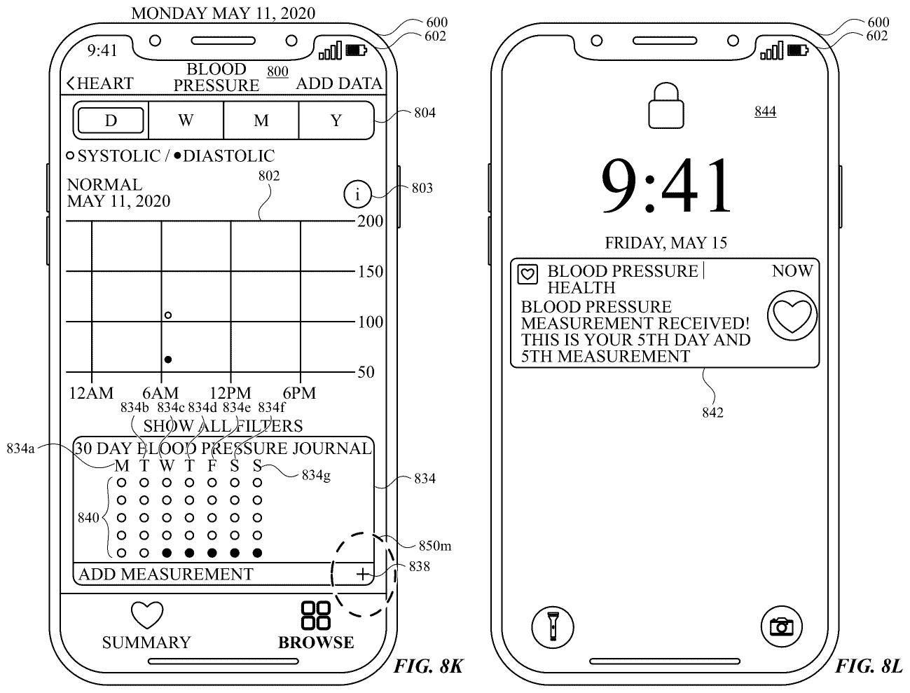

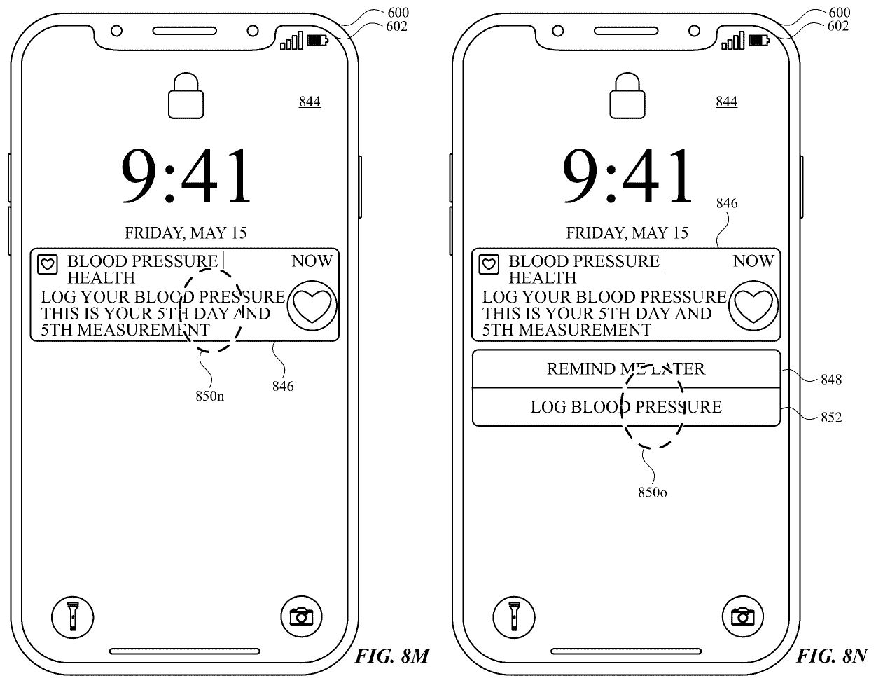

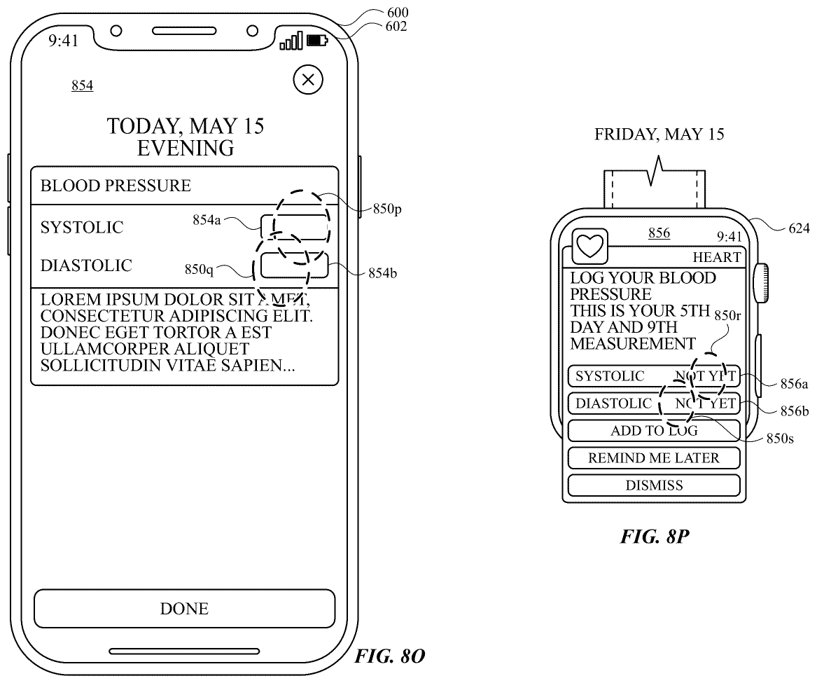

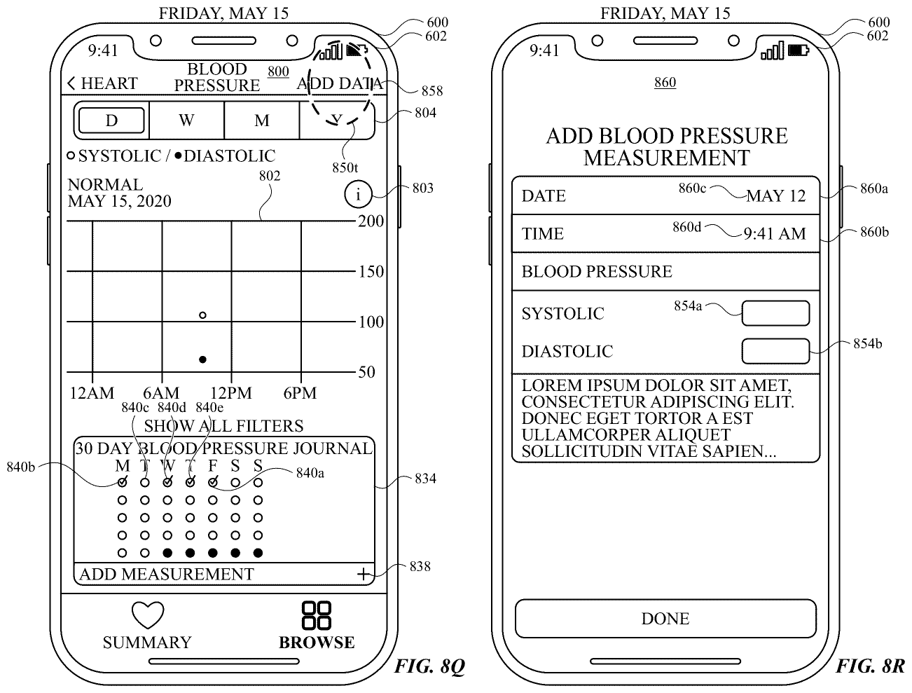

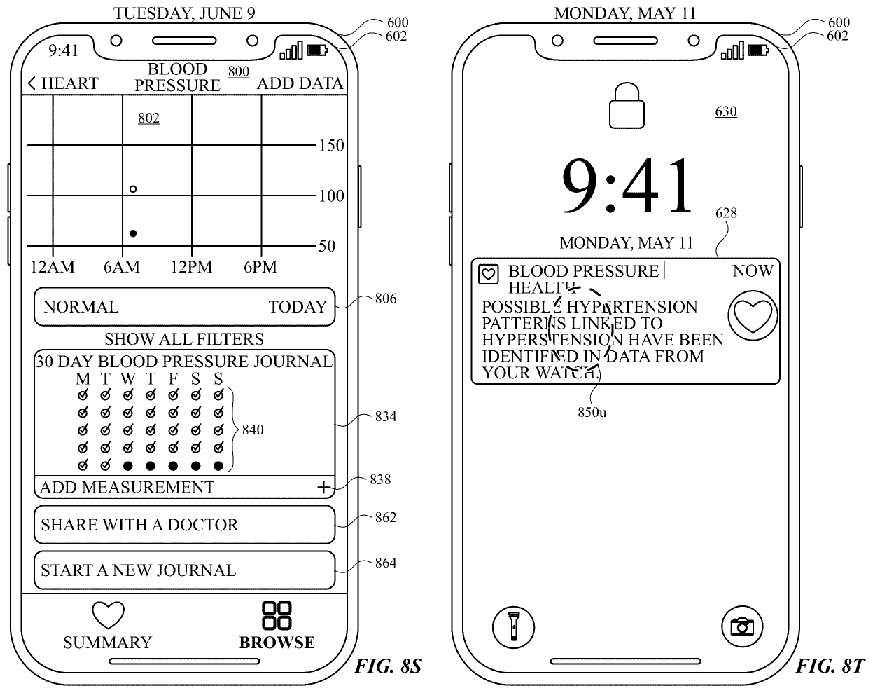

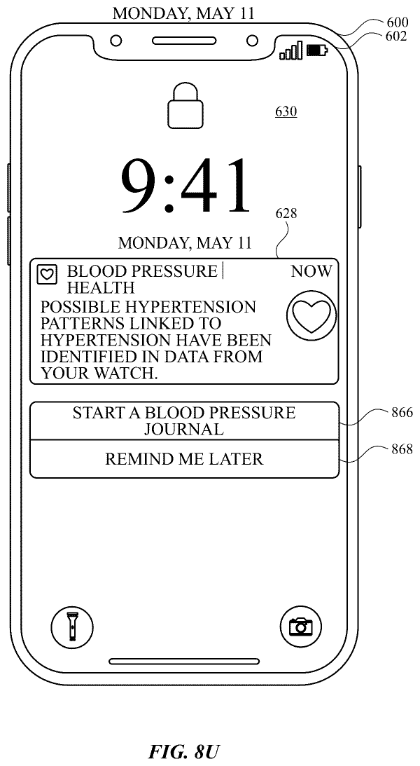

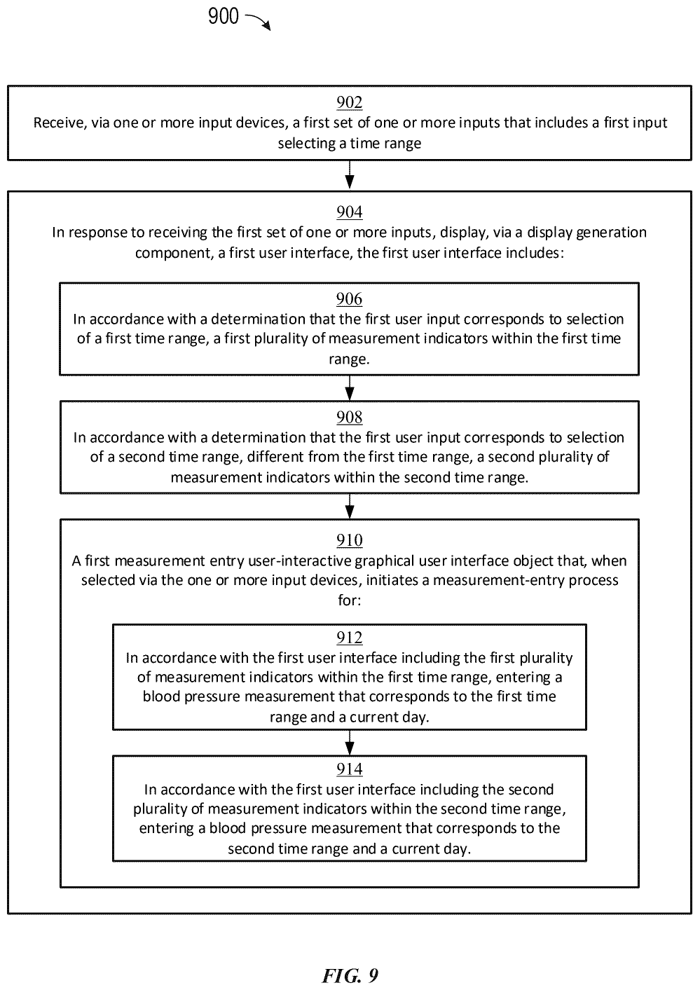

Below, FIGS. 1A-1B, 2, 3, 4A-4B, and 5A-5B provide a description of exemplary devices for performing the techniques for managing event notifications. FIGS. 6A-6P illustrate exemplary user interfaces for generating a notification associated with a health event. FIG. 7 is a flow diagram illustrating methods of generating a notification associated with a health event in accordance with some embodiments. The user interfaces in FIGS. 6A-6P are used to illustrate the processes described below, including the processes in FIG. 7. FIGS. 8A-8U illustrate exemplary user interfaces for managing health information over a predefined time range. FIG. 9 is a flow diagram illustrating methods of managing health information over a predefined time range in accordance with some embodiments. The user interfaces in FIGS. 8A-8U are used to illustrate the processes described below, including the processes in FIG. 9.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

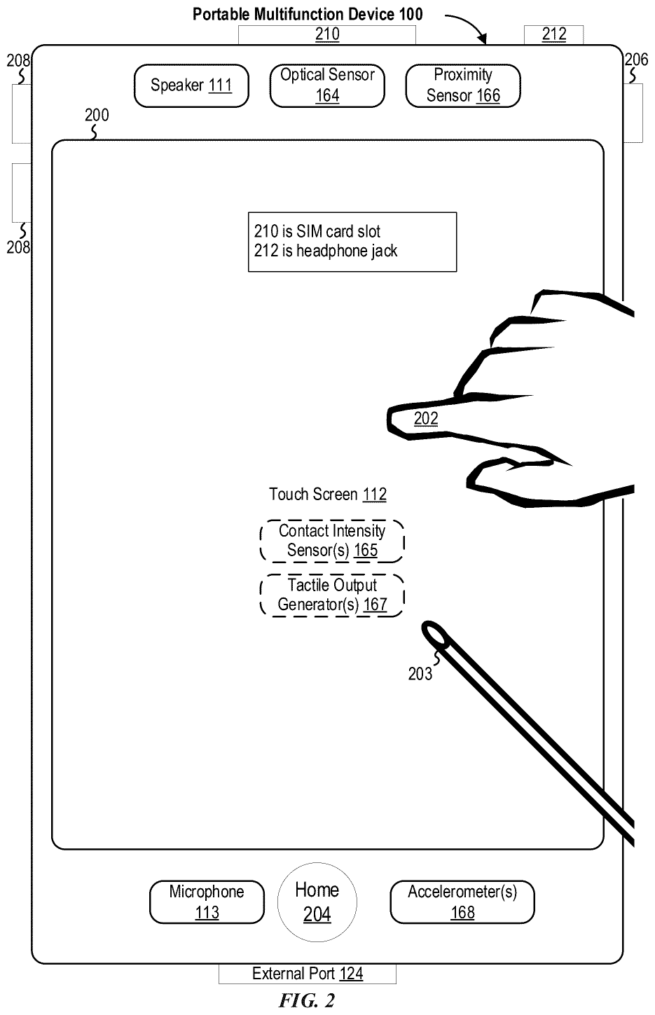

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

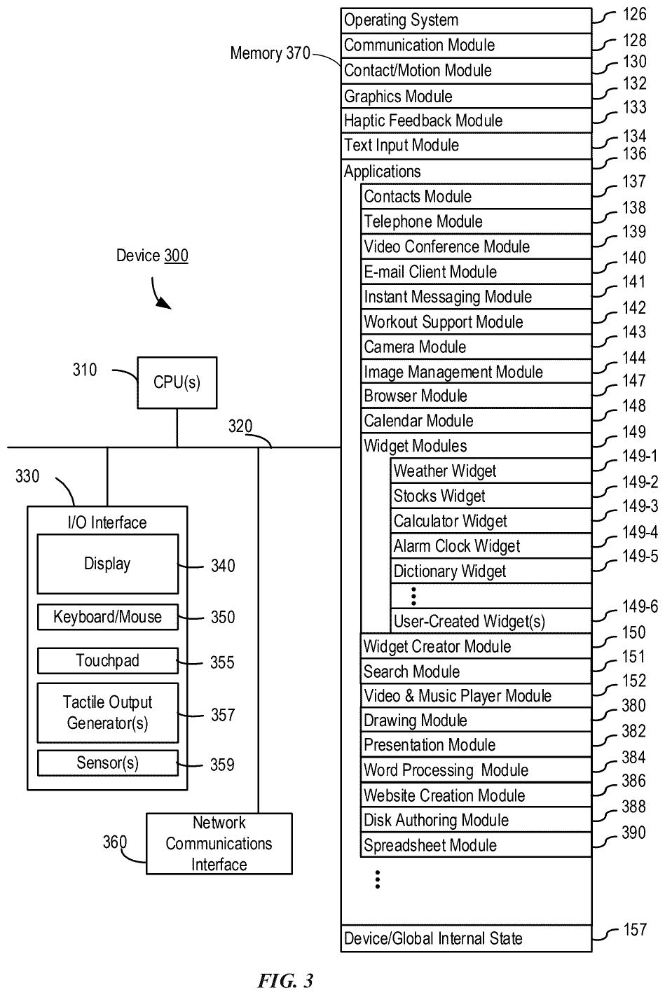

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (187) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definition 187 includes a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

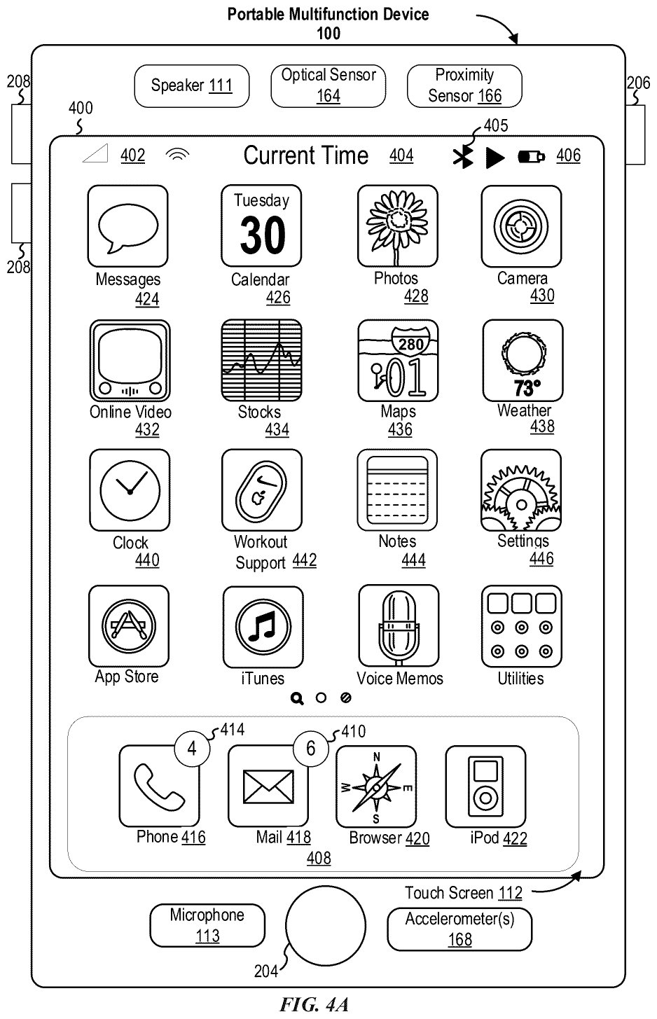

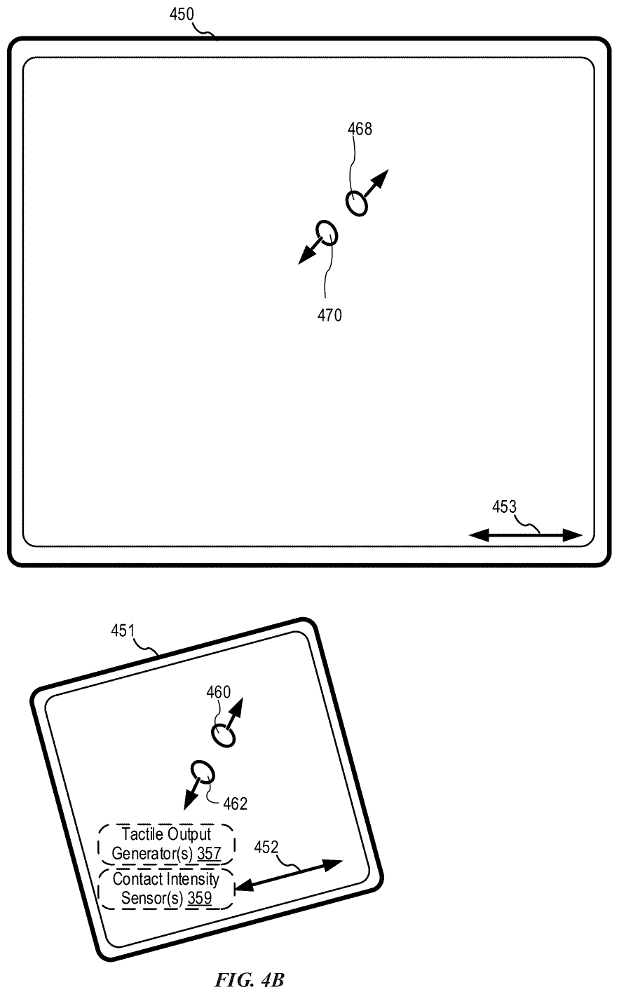

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch-sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contact 462 corresponds to 470). In this way, user inputs (e.g., contacts 460 and 462, and movements thereof) detected by the device on the touch-sensitive surface (e.g., touch-sensitive surface 451 in FIG. 4B) are used by the device to manipulate the user interface on the display (e.g., display 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

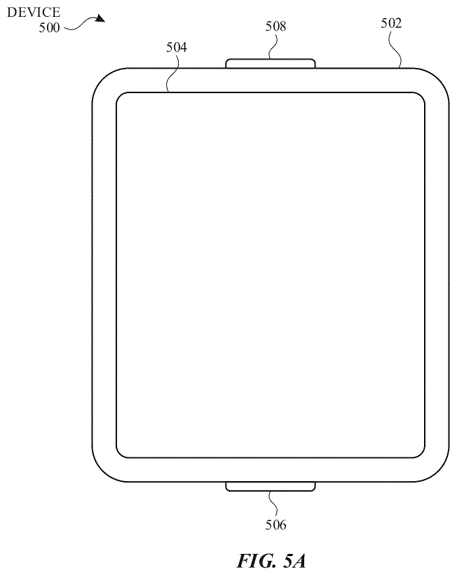

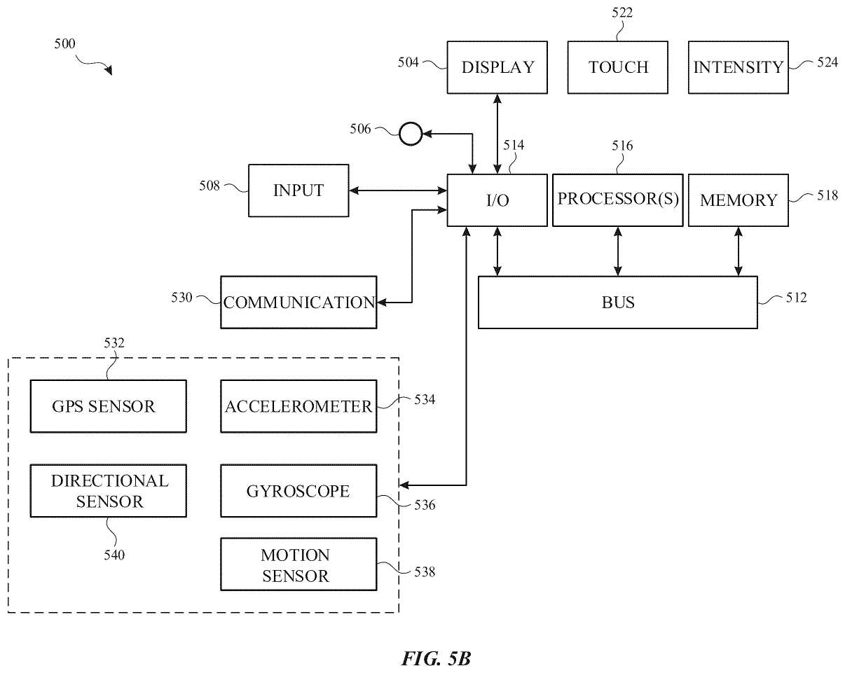

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700 and 900 (FIGS. 7 and 9). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

In some embodiments, a portion of a gesture is identified for purposes of determining a characteristic intensity. For example, a touch-sensitive surface optionally receives a continuous swipe contact transitioning from a start location and reaching an end location, at which point the intensity of the contact increases. In this example, the characteristic intensity of the contact at the end location is, optionally, based on only a portion of the continuous swipe contact, and not the entire swipe contact (e.g., only the portion of the swipe contact at the end location). In some embodiments, a smoothing algorithm is, optionally, applied to the intensities of the swipe contact prior to determining the characteristic intensity of the contact. For example, the smoothing algorithm optionally includes one or more of: an unweighted sliding-average smoothing algorithm, a triangular smoothing algorithm, a median filter smoothing algorithm, and/or an exponential smoothing algorithm. In some circumstances, these smoothing algorithms eliminate narrow spikes or dips in the intensities of the swipe contact for purposes of determining a characteristic intensity.

The intensity of a contact on the touch-sensitive surface is, optionally, characterized relative to one or more intensity thresholds, such as a contact-detection intensity threshold, a light press intensity threshold, a deep press intensity threshold, and/or one or more other intensity thresholds. In some embodiments, the light press intensity threshold corresponds to an intensity at which the device will perform operations typically associated with clicking a button of a physical mouse or a trackpad. In some embodiments, the deep press intensity threshold corresponds to an intensity at which the device will perform operations that are different from operations typically associated with clicking a button of a physical mouse or a trackpad. In some embodiments, when a contact is detected with a characteristic intensity below the light press intensity threshold (e.g., and above a nominal contact-detection intensity threshold below which the contact is no longer detected), the device will move a focus selector in accordance with movement of the contact on the touch-sensitive surface without performing an operation associated with the light press intensity threshold or the deep press intensity threshold. Generally, unless otherwise stated, these intensity thresholds are consistent between different sets of user interface figures.

An increase of characteristic intensity of the contact from an intensity below the light press intensity threshold to an intensity between the light press intensity threshold and the deep press intensity threshold is sometimes referred to as a “light press” input. An increase of characteristic intensity of the contact from an intensity below the deep press intensity threshold to an intensity above the deep press intensity threshold is sometimes referred to as a “deep press” input. An increase of characteristic intensity of the contact from an intensity below the contact-detection intensity threshold to an intensity between the contact-detection intensity threshold and the light press intensity threshold is sometimes referred to as detecting the contact on the touch-surface. A decrease of characteristic intensity of the contact from an intensity above the contact-detection intensity threshold to an intensity below the contact-detection intensity threshold is sometimes referred to as detecting liftoff of the contact from the touch-surface. In some embodiments, the contact-detection intensity threshold is zero. In some embodiments, the contact-detection intensity threshold is greater than zero.

In some embodiments described herein, one or more operations are performed in response to detecting a gesture that includes a respective press input or in response to detecting the respective press input performed with a respective contact (or a plurality of contacts), where the respective press input is detected based at least in part on detecting an increase in intensity of the contact (or plurality of contacts) above a press-input intensity threshold. In some embodiments, the respective operation is performed in response to detecting the increase in intensity of the respective contact above the press-input intensity threshold (e.g., a “down stroke” of the respective press input). In some embodiments, the press input includes an increase in intensity of the respective contact above the press-input intensity threshold and a subsequent decrease in intensity of the contact below the press-input intensity threshold, and the respective operation is performed in response to detecting the subsequent decrease in intensity of the respective contact below the press-input threshold (e.g., an “up stroke” of the respective press input).

In some embodiments, the device employs intensity hysteresis to avoid accidental inputs sometimes termed “jitter,” where the device defines or selects a hysteresis intensity threshold with a predefined relationship to the press-input intensity threshold (e.g., the hysteresis intensity threshold is X intensity units lower than the press-input intensity threshold or the hysteresis intensity threshold is 75%, 90%, or some reasonable proportion of the press-input intensity threshold). Thus, in some embodiments, the press input includes an increase in intensity of the respective contact above the press-input intensity threshold and a subsequent decrease in intensity of the contact below the hysteresis intensity threshold that corresponds to the press-input intensity threshold, and the respective operation is performed in response to detecting the subsequent decrease in intensity of the respective contact below the hysteresis intensity threshold (e.g., an “up stroke” of the respective press input). Similarly, in some embodiments, the press input is detected only when the device detects an increase in intensity of the contact from an intensity at or below the hysteresis intensity threshold to an intensity at or above the press-input intensity threshold and, optionally, a subsequent decrease in intensity of the contact to an intensity at or below the hysteresis intensity, and the respective operation is performed in response to detecting the press input (e.g., the increase in intensity of the contact or the decrease in intensity of the contact, depending on the circumstances).

For ease of explanation, the descriptions of operations performed in response to a press input associated with a press-input intensity threshold or in response to a gesture including the press input are, optionally, triggered in response to detecting either: an increase in intensity of a contact above the press-input intensity threshold, an increase in intensity of a contact from an intensity below the hysteresis intensity threshold to an intensity above the press-input intensity threshold, a decrease in intensity of the contact below the press-input intensity threshold, and/or a decrease in intensity of the contact below the hysteresis intensity threshold corresponding to the press-input intensity threshold. Additionally, in examples where an operation is described as being performed in response to detecting a decrease in intensity of a contact below the press-input intensity threshold, the operation is, optionally, performed in response to detecting a decrease in intensity of the contact below a hysteresis intensity threshold corresponding to, and lower than, the press-input intensity threshold.

Attention is now directed towards embodiments of user interfaces (“UI”) and associated processes that are implemented on an electronic device, such as portable multifunction device 100, device 300, or device 500.

FIGS. 6A-6P illustrate exemplary user interfaces for generating notifications associated with a health event, in accordance with some embodiments. The user interfaces in these figures are used to illustrate the processes described below, including the processes in FIG. 7.

FIG. 6A illustrates computer system 600 having display generation component 602 and displaying, via display generation component 602, health user interface 604 of a health application of computer system 600 on a first date (e.g., Sunday, April 5th). At FIG. 6A, health user interface 604 includes health data user interface objects 604a and 604b and health event notification user interface object 606. Health data user interface objects 604a and 604b include information related to health parameters (e.g., quantitative and/or qualitative measurements and/or characteristics detected by a sensor of computer system 600 and/or received by computer system 600 via user input and/or from an external device) of a user associated with computer system 600. In some embodiments, health user interface 604 is a home screen or a default screen of the health application that computer system 600 displays, via display generation component 602, upon launching the health application.

At FIG. 6A, computer system 600 displays health event notification user interface object 606 prompting a user to enable and/or activate notifications related to a particular health event. Health event notification user interface object 606 is associated with hypertension notifications that computer system 600 generates in response to a determination that a user associated with computer system 600 is at risk for hypertension. As used herein, “hypertension” refers to a user having and/or being at risk of chronic high blood pressure, as opposed to a single instance and/or single measurement of high blood pressure. Therefore, computer system 600 generates hypertension notifications upon determining that a user associated with computer system 600 has regular occurrences of high blood pressure (e.g., instead of the user simply having one instance and/or one measurement indicative of high blood pressure).

Computer system 600 displays health event notification user interface object 606 on health user interface 604 based on a determination that the user has not previously enabled and/or activated hypertension notifications. In some embodiments, health event notification user interface object 606 can be displayed on another user interface of the health application, such as a checklist user interface that prompts a user to enable and/or activate tracking of health parameters (e.g., heart rate and/or rhythm monitoring, electrocardiogram monitoring, blood oxygen monitoring, noise exposure monitoring, and/or cardio fitness level monitoring) that have not yet been enabled and/or activated by the user. In some embodiments, computer system 600 enables and/or activates hypertension notifications by default and/or without user input (e.g., computer system 600 enables hypertension notifications as a default setting upon initial start-up of computer system 600).

At FIG. 6A, computer system 600 detects user input 650a (e.g., a tap gesture) corresponding to selection of user interface object 606a (e.g., user interface object labeled “Enable”) of health event notification user interface object 606. In response to detecting user input 650a, computer system 600 displays first set-up user interface 608 on the first date, as shown at FIG. 6B. First set-up user interface 608 includes additional information related to enabling and/or activating hypertension notifications. For instance, first set-up user interface 608 includes learn more region 608a that provides information related to hypertension and/or the hypertension notifications. As such, the user associated with computer system 600 can obtain further guidance from learn more region 608a before deciding to enable and/or activate the hypertension notifications. At FIG. 6B, computer system 600 detects user input 650b (e.g., a tap gesture) on user interface object 608b (e.g., user interface object labeled “Set Up”). In response to detecting user input 650b, computer system 600 displays second set-up user interface 610 on the first date, as shown at FIG. 6C.

Second set-up user interface 610 includes further information (e.g., textual information) regarding the hypertension notifications that computer system 600 generates. At FIG. 6C, computer system 600 detects user input 650c (e.g., a tap gesture) on user interface object 610a (e.g., user interface object labeled “Enable”) of second set-up user interface 610. In response to detecting user input 650c, computer system 600 displays third set-up user interface 612 on the first date, as shown at FIG. 6D.

Third set-up user interface 612 includes birth date user interface object 612a, first diagnosis user interface object 612b, second diagnosis user interface object 612c, and next user interface object 612d. In some embodiments, computer system 600 is configured to enable and/or activate the hypertension notifications for users that satisfy feature-eligibility criteria and to not enable and/or activate (e.g., block enabling and/or activation of) the hypertension notifications for user that do not satisfy the feature-eligibility criteria. For example, the feature-eligibility criteria includes a criterion that the user is above a threshold age (e.g., 22 years old). Accordingly, computer system 600 receives information related to a birth date of the user associated with computer system 600. In some embodiments, computer system 600 displays (e.g., pre-populates) a birth date in birth date user interface object 612a corresponding to a birth date previously received by computer system 600 (e.g., input by the user and/or received by computer system 600 during an initial set up and/or activation of computer system 600). In some embodiments, computer system 600 receives the birth date and displays the received birth date in birth date user interface object 612a in response to one or more inputs corresponding to selection of birth date user interface object 612a.

In addition, the feature-eligibility criteria includes a criterion that the user has not been diagnosed with hypertension (e.g., received a diagnosis from a physician). At FIG. 6D, third set-up user interface 612 includes first diagnosis user interface object 612b corresponding to the user having been previously diagnosed with hypertension (e.g., the user obtained a hypertension diagnosis from a physician). Second diagnosis user interface object 612c corresponds to the user not having been previously diagnosed with hypertension (e.g., the user has not obtained a hypertension diagnosis from a physician). In some embodiments, computer system 600 is configured to enable and/or activate the hypertension notifications for users that have not received a hypertension diagnosis and configured to not enable and/or activate (e.g., block enabling and/or activation of) hypertension notifications for users that have received a hypertension diagnosis. In some embodiments, computer system 600 is configured to not enable and to not collect and store sensor data for the purposes of determining whether to provide hypertension notifications for users that do not meet the feature-eligibility criteria. In some embodiments, computer system 600 does not make determinations based on hypertension notification criteria for users that do not meet the feature-eligibility criteria.

In some embodiments, next user interface object 612d is not available for selection (e.g., via user input) in response to the birth date being associated with an age below the threshold age and/or in response to user input selecting first diagnosis user interface object 612b (e.g., next user interface object 612d is greyed out or not displayed). In other words, computer system 600 does not enable the user to complete the set-up (e.g., enabling and/or activation) of hypertension notifications if the user is below the threshold age and/or the user has previously been diagnosed with hypertension. In some embodiments, computer system 600 forgoes display of health event notification user interface object 606 on health user interface 604 in response to detecting that an age of the user is below the threshold age and/or in response to receiving data indicative of a hypertension diagnosis of the user associated with computer system 600.

At FIG. 6D, computer system 600 receives and/or detects a birth date of the user that is associated with an age above the threshold age and receives user input corresponding to selection of second diagnosis user interface object 612c. As such, next user interface object 612d is available for selection. At FIG. 6D, computer system 600 detects user input 650d on next user interface object 612d. In response to detecting user input 650d, computer system 600 displays fourth set-up user interface 614 on the first date, as shown at FIG. 6E.

At FIG. 6E, fourth set-up user interface 614 includes first informational platter 614a, second informational platter 614b, and third informational platter 614c. Informational platters 614a, 614b, and 614c provided additional details regarding the hypertension notifications. The additional details may include textual information that provides guidance and/or suggested actions for the user upon receipt and/or absence of a hypertension notification. At FIG. 6E, computer system 600 detects user input 650e on user interface object 614d of fourth set-up user interface 614. In response to detecting user input 650e, computer system 600 displays confirmation user interface 616 on the first date, as shown at FIG. 6F.

At FIG. 6F, confirmation user interface 616 includes hypertension notifications user interface object 616a (e.g., a toggle). Hypertension notifications user interface object 616a is in an active and/or “on” position, thereby indicating that computer system 600 successfully enabled and/or activated the hypertension notifications. Confirmation user interface 616 verifies to the user that the set-up process for enabling the hypertension notifications is successfully completed. In some embodiments, computer system 600 detects user input (e.g., a tap gesture; not shown) on done user interface object 616b. In response to detecting user input on done user interface object 616b, computer system 600 displays health user interface 604 (e.g., health user interface 604 without health event notification user interface object 606) and/or hypertension user interface 644 (e.g., hypertension user interface 644 without notification user interface objects 646a and 646b), as described in below with reference to FIG. 6L.

After computer system 600 enables and/or activates the hypertension notifications, computer system 600 initiates a process for one or more sensors 618, 620, and/or 622 of computer system 624 to collect data corresponding to one or more parameters and/or characteristics of user of computer system 624. As set forth below, computer system 624 is in communication with computer system 600 and provides (e.g., transmits) collected data and/or an indication that a set of notification criteria (e.g., hypertension notification criteria) has been satisfied to computer system 600 (e.g., via a wireless signal, such as Bluetooth, Wi-Fi, and/or Zigbee).

At FIG. 6G, computer system 624 collects data from sensors 618, 620, and/or 622 over a predetermined period of time, such as 30 days, before computer system 624 and/or computer system 600 determines whether the data satisfies the set of notification criteria (e.g., hypertension notification criteria). For instance, FIG. 6G includes table 626 to illustrate days and/or times at which data is collected from sensors 618, 620, and/or 622 after computer system 600 enables and/or activates the hypertension notifications. Table 626 includes first day 626a, second day 626b, and third day 626c that each fall within the predetermined period of time (e.g., 30 days from the first date of Sunday, April 5th). As shown at FIG. 6G, data is collected on each of first day 626a, second day 626b, and third day 626c as represented by check marks in the “Data Collected” column of table 626. While FIG. 6G illustrates that data is collected on first day 626a, second day 626b, and third day 626c within the predetermined period of time, data is also collected at other times (e.g., other days and/or multiple times within first day 626a, second day 626b, and/or third day 626c) within the predetermined period of time. In some embodiments, data is collected on days that the user wears computer system 624 (e.g., on a wrist of user) for an amount of time that is sufficient for sensors 618, 620, and/or 622 to collect at least one measurement. In some embodiments, computer system 600 and/or computer system 624 determine whether the data satisfies the set of notification criteria (e.g., hypertension notification criteria) in response to the data including a threshold number of data points. For example, computer system 600 and/or computer system 624 determines whether the data includes data points collected on at least 15 days of the 30 day period of time and/or that the data includes data points for at least 5 hours, 8 hours, 10 hours, and/or 12 hours for each of the 15 days within the 30 day time period before making the determination as to whether or not the data satisfies the notification criteria. In some embodiments, computer system 600 and/or computer system 624 does not determine whether the data satisfies the set of notification criteria (e.g., hypertension notification criteria) if the data does not include the threshold number of data points. As such, computer system 600 and/or computer system 624 does not generate a hypertension notification unless the data includes a threshold number of data points.

The predetermined period of time is sufficiently long so that computer system 624 collects enough data to determine whether the user is at risk for hypertension. Therefore, the predetermined period of time is greater than 1 hour, 1 day, and/or 1 week. Further, the data collected over the predetermined period of time includes individual data points that are spaced apart from one another by an amount of time that is greater than an amount of time for collecting a respective data point. For example, the data collected over the period of time can include a first data point collected on a first day of the predetermined period of time and a second data point collected on a second day of the predetermined period of time, where the first day and the second day are spaced apart by multiple days. In addition, sensors 618, 620, and/or 622 can collect individual data points in less than 1 second, 1 second, and/or 1 minute. Thus, the time computer system 624 spends collecting individual data points is substantially less than the predetermined period of time over which data is ultimately collected in order to determine whether the data satisfies the set notification criteria.

As set forth above, computer system 600 and/or computer system 624 determines whether the data collected over the predetermined period of time satisfies the set of notification criteria after the predetermined period of time has elapsed. In some embodiments, the set of notification criteria includes a threshold risk score (e.g., a quantitative value). Computer system 600 and/or computer system 624 determines a risk score by inputting the data collected over the predetermined period of time into an algorithm (e.g., a machine learning algorithm). The algorithm is configured to assess the data collected over the predetermined period of time for trends and/or patterns associated with hypertension. For instance, the algorithm is generated based on clinical data from users other than the current user associated with computer system 600 and computer system 624. The clinical data includes data from users diagnosed with hypertension, and optionally, data from users that have normal blood pressure and have not been diagnosed with hypertension. Accordingly, the algorithm analyzes the data in order to generate a risk score (e.g., a quantitative value) associated with whether the user associated with computer system 600 and computer system 624 has hypertension. In response to the algorithm generating a risk score that meets or exceeds the threshold risk score, computer system 600 and/or computer system 624 determines that the data collected over the predetermined period of time satisfies the set of notification criteria. In contrast, in response to the algorithm generating a risk score that is below the threshold risk score, computer system 600 and/or computer system 624 determines that the data collected over the predetermined period of time does not satisfy the set of notification criteria.

At FIG. 6G, sensor 618 is a photoplethysmogram sensor (e.g., a photoplethysmograph) that collects data indicative of changes in light absorption of the skin. For instance, sensor 618 of computer system 624 emits light 618a, which reflects off of skin of the user associated with computer system 624 (e.g., the user wearing computer system 624 and the same user associated with computer system 600). Light 618a is reflected from the skin of the user and received by sensor 618 of computer system 624, which may enable computer system 624 (and/or computer system 600) to determine changes in blood volume of the user associated with computer system 624. In addition, sensor 620 is a gyro sensor and/or an accelerometer that collects data indicative of movement (e.g., angular velocity) 620a of the user associated with computer system 624. Further still, sensor 622 is an electrocardiogram (ECG) sensor and/or a heart rate sensor that collects data indicative of a heart rate of the user associated with computer system 624.

Accordingly, sensors 618, 620, and 622 of computer system 624 do not directly measure pressure and/or blood pressure of the user associated with computer system 624. In particular, sensors 618, 620, and 622 do not provide computer system 624 and/or computer system 600 with raw data that includes pressure and/or blood pressure. Further, computer system 624 and/or sensors 618, 620, and 622 of computer system 624 do not apply pressure to, and/or restrict blood flow of, the user associated with computer system 624. Further still, sensors 618, 620, and 622 do no measure vibration. Therefore, sensors 618, 620, and 622, either alone or collectively, are not traditional blood pressure measurement devices, such as a sphygmomanometer, which measures pressure, vibration, and/or acoustics associated with blood flowing through arteries and/or blood vessels. Instead, sensors 618, 620, and 622 provide data associated with light absorption of the skin of the user, movement of the user, and/or a heart rate of the user to computer system 624 and/or computer system 600. That data received from sensors 618, 620, and 622 is then input into an algorithm to determine the risk score of the data collected over the predetermined period, which is then compared to the threshold risk score.

Further, the data collected by sensors 618, 620, and 622 is passively measured. In other words, computer system 624 causes sensors 618, 620, and 622 to collect the data while the user wears computer system 624 and without the user providing an input each time a data point is measured. Therefore, the user associated with computer system 624 (e.g., the same user associated with computer system 600) can enable hypertension notifications via the set-up process described above with respect to FIGS. 6A-6F and not provide any further inputs to computer system 600 and/or computer system 624 in order for computer system 600 and/or computer system 624 to collect the data over the predetermined period of time.

As set forth above, data measured and/or collected from sensors 618, 620, and 622 is received by computer system 624 and/or computer system 600 (e.g., via computer system 624). Computer system 624 and/or computer system 600 utilizes the data collected over the predetermined period of time to determine whether the set of notification criteria is met (e.g., whether the risk score generated by the algorithm using the data exceeds the threshold risk score). In response to determining that the data collected over the predetermined period of time satisfies the set of notification criteria, computer system 600 and/or computer system 624 generate a notification indicating that the user associated with computer system 600 and computer system 624 is at risk for hypertension.

At FIG. 6H, computer system 600 displays notification 628 on user interface 630 (e.g., a lock screen user interface and/or a home screen user interface). Computer system 600 displays notification 628 on a second date (e.g., Tuesday, May 5th), which is a date that occurs after the predetermined period of time has elapsed starting from the first date (e.g., Sunday, April 5th). Accordingly, computer system 600 (and/or computer system 624) determines that the data collected during the predetermined period of time (e.g., as shown at FIG. 6G) satisfies the set of notification criteria, thereby causing display of notification 628. At FIG. 6H, notification 628 includes application indicator 628a and information indicator 628b. Application indicator 628a includes graphical user interface elements and/or text indicative of an application (e.g., a health application) of computer system 600 that is associated with notification 628. In addition, information indicator 628b provides details (e.g., textual details) indicating that the notification 628 pertains to the user being at risk for hypertension. As shown at FIG. 6H, notification 628 does not include an indication of a blood pressure measurement of the user (e.g., a numeric indicator and/or quantitative value of systolic and/or diastolic pressure), but instead notifies the user that data collected indicates that the user is at risk for hypertension.

In some embodiments, computer system 600 and/or computer system 624 reset the predetermined period of time after generating notification 628. As such, computer system 600 and/or computer system 624 does not generate another notification indicating that the user is at risk of hypertension until collecting another set of data within a second predetermined period of time (e.g., 30 days; a predetermined period of time that does not overlap with the predetermined period of time used to generate notification 628). For example, computer system 600 generates and displays notification 628 on Tuesday, May 5th. Computer system 600 and/or computer system 624 resets the predetermined period of time, such that a second notification is not generated and/or displayed until after the predetermined period of time elapses again starting from Tuesday, May 5th. In other words, the predetermined period of time for collecting data that is used to generate a first hypertension notification does not overlap with the predetermined period of time for collecting data that is used to generate a second hypertension notification.

In particular, at FIG. 6I, computer system 600 displays notification 632 on user interface 630 (e.g., a lock screen user interface). Computer system 600 displays notification 632 on a third date (e.g., Friday, June 5th), which occurs after the predetermined period of time (e.g., 30 days) has elapsed starting from the second date (e.g., Tuesday, May 5th). As such, computer system 600 and/or computer system 624 resets the predetermined period of time in response to generating and displaying notification 628, such that a subsequent notification (e.g., notification 632) is not generated and/or displayed until after the predetermined period of time has elapsed starting from the time notification 628 was generated. At FIG. 6I, notification 632 includes application indicator 632a and information indicator 632b. Application indicator 632a includes graphical user interface elements and/or text indicating an application (e.g., a health application) of computer system 600 that is associated with notification 632. In addition, information indicator 632b provides details (e.g., textual details) indicating that the notification 632 pertains to the user being at risk for hypertension. As shown at FIG. 6I, notification 632 does not include an indication of a blood pressure measurement of the user (e.g., a numeric indicator and/or quantitative value of systolic and/or diastolic pressure), but instead notifies the user that data collected indicates that the user is at risk for hypertension.

At FIG. 6I, computer system 600 detects user input 650f (e.g., a tap gesture and/or user input satisfying a set of unlock criteria) on notification 632. In response to detecting user input 650f, computer system 600 displays health event user interface 634, as shown at FIG. 6J. Health event user interface 634 corresponds to notification 632 and provides further information and/or details associated with notification 632. Similar to notification 632, health event user interface 634 does not include a blood pressure measurement of the user, such as a numeric and/or quantitative value of a systolic and/or a diastolic pressure measurement. Instead, health event user interface 634 includes information region 634a, notification details region 636, and device details region 638. Information region 634a provides textual indications related to the cause for computer system 600 displaying notification 632 and additional information for the user to consider as a result of the notification 632. Notification details region 636 includes date user interface object 636a indicating a date and time that notification 632 was generated by computer system 600 (and/or computer system 624), source user interface object 636b indicating the application of computer system 600 associated with notification 632 (e.g., the health application), and log user interface object 636c indicating a date and time that notification was logged, stored, and/or otherwise recorded by the application indicated by source user interface object 636b. Device details region 638 provides one or more user interface objects indicative of a particular device (e.g., computer system 600 and/or computer system 624) that generated notification 632.

In some embodiments, the user of computer system 600 can access health event user interface 634 via health user interface 604 in addition to, or in lieu of, user input 650f. For instance, at FIG. 6K, computer system 600 displays health user interface 604, which includes notification 640. Notification 640 corresponds to notification 632, and thus, computer system 600 displays notification 640 in response to a determination (e.g., by computer system 600 and/or computer system 624) that data collected over the predetermined period of time satisfies the set of notification criteria for generating a hypertension notification. At FIG. 6K, notification 640 includes an indication (e.g., textual indication) providing the user with information related to the cause of notification 640. In addition, notification 640 includes learn more user interface object 640a. In response to detecting user input corresponding to selection of learn more user interface object 640a, computer system 600 displays an educational user interface that provides the user with further information related to hypertension and suggested actions that the user can take as a result of receiving notification 640.

Health user interface 604 of FIG. 6K is translated in an upwards direction as compared to health user interface 604 of FIG. 6A. As such, computer system 600 displays notifications, such as notification 640, in a notification region 642 of health user interface 604, where the notifications in notification region 642 are associated with and/or generated by the health application. In some embodiments, notifications region 642 of health user interface 604 is positioned above and/or before health data user interface objects 604a and/or 604b when computer system 600 launches the health application and displays health user interface 604 (e.g., before receiving a user input requesting to translate health user interface 604). Accordingly, the user associated with computer system 600 can easily view recent notifications corresponding to health events and/or health information that are relevant to the user when the health application is launched by computer system 600.

At FIG. 6K, computer system 600 detects user input 650g (e.g., a tap gesture) corresponding to selection of notification 640. In response to detecting user input 650g, computer system 600 displays hypertension user interface 644, as shown at FIG. 6L. Hypertension user interface 644 is another user interface associated with the health application of computer system 600, but includes user interface objects that are specifically related to the hypertension notifications and information associated with hypertension and/or the hypertension notifications. For example, hypertension user interface 644 includes status user interface object 644a, notifications region 646, and learn more region 648. Status user interface object 644a provides an indication as to the status of the hypertension notifications feature (e.g., whether computer system 600 has enabled and/or activated the hypertension notifications). In some embodiments, status user interface object 644a includes an indication (e.g., a textual indication) of a date and/or time when data from sensors 618, 620, and 622 was last collected within the predetermined period of time.

Notifications region 646 of hypertension user interface 644 includes first notification user interface object 646a and second notification user interface object 646b. First notification user interface object 646a corresponds to notification 632 generated on the third date (e.g., June 5th) and second notification user interface object 646b corresponds to notification 628 generated on the second date (e.g., May 5th). As such, computer system 600 displays a history of the hypertension notifications generated by computer system 600 and/or computer system 624 within notifications region 646 of hypertension user interface 644. Thus, the user associated with computer system 600 and computer system 624 can access and view a history of the hypertension notifications that have been generated by computer system 600 and/or computer system 624. In some embodiments, notifications region 646 includes more notification user interface objects than the two notification user interface objects 646a and 646b (e.g., when additional hypertension notifications have been generated by computer system 600 and/or computer system 624). In some embodiments, computer system 600 displays the notification user interface objects within notifications region 646 in reverse chronological order (e.g., most recent at the top and least recent at the bottom). In some embodiments, computer system 600 displays a predetermined number of notification user interface objects in notification region 646 and displays a user interface object (e.g., “See All”) that, when selected, causes computer system 600 to display a list of notification user interface objects for all hypertension notifications generated by computer system 600 and/or computer system 624. In some embodiments, computer system 600 adjusts a size of notifications region 646 on hypertension user interface 644, such that notification user interface objects corresponding to each respective hypertension notification is displayed on hypertension user interface 644.

In some embodiments, computer system 600 and/or computer system 624 determines that the data collected over the predetermined period of time does not satisfy the set of notification criteria. In response to determining that the data collected over the predetermined period of time does not satisfy the set of notification criteria, computer system 600 forgoes display of notifications 628 and 632, as well as forgoes display of notification user interface objects 646a and 646b on hypertension user interface 644.

At FIG. 6L, computer system 600 detects user input 650h (e.g., a tap gesture) corresponding to selection of first notification user interface object 646a. In response to detecting user input 650h, computer system 600 displays health event user interface 634, as shown at FIG. 6J, which corresponds to notification 632 generated by computer system 600 and/or computer system 624 on the third date (e.g., June 5th). In some embodiments, computer system 600 displays health user interface 634 corresponding to notification 628 generated by computer system 600 and/or computer system 624 on the second date (e.g., May 5th) in response to user input corresponding to selection of second notification user interface object 646b.

In addition, at FIG. 6L, learn more region 648 of hypertension user interface 644 includes first user interface object 648a (e.g., labeled “Things to Know”), second user interface object 648b (e.g., labeled “How Hypertension Notifications Work”), and third user interface object 648c (e.g., labeled “Learn More” and/or “About Hypertension”). In response to user input corresponding to selection of the user interface objects 648a, 648b, and/or 648c, computer system 600 displays an educational user interface that provides the user with additional details and/or information related to hypertension, the hypertension notifications, and/or suggested actions for the user as a result of receiving a hypertension notification.

At FIG. 6L, computer system 600 detects user input 650i (e.g., a swipe gesture; an upward swipe gesture) on hypertension user interface 644. In response to detecting user input 650i, computer system 600 translates hypertension user interface 644 and displays additional options region 652 of hypertension user interface 644 (e.g., displays additional options region 652 concurrently with learn more region 648 and ceases to display notifications region 646), as shown at FIG. 6M. At FIG. 6M, additional options region 652 includes favorites user interface object 652a, all data user interface object 652b, and source user interface object 652c.

In response to user input corresponding to selection of favorites user interface object 652a, computer system 600 is configured to add a user interface object to health user interface 604 that is displayed in a favorites region (e.g., a region of health user interface 604 that includes health data user interface objects 604a and/or 604b). As such, information related the hypertension notifications may be included on health user interface 604 after the hypertension notifications are enabled and/or activated and regardless of whether a hypertension notification was recently generated (e.g., by computer system 600 and/or computer system 624).

In response to user input corresponding to all data user interface object 652b, computer system 600 displays a list of dates and/or times at which data was collected and/or measured by sensors 618, 620, and 622 of computer system 624. In some embodiments, computer system 600 displays additional information (e.g., a measurement value(s), a device used to collect the data, a date and/or time at which the data was added to the health application, and/or a test type) in response to user input corresponding to selection of an item of the list of dates and/or times at which data was collected.

In response to user input corresponding to source user interface object 652c, computer system 600 displays an additional user interface having information related to computer systems and/or devices used to collect and/or measure data for use in determining whether to generate the hypertension notifications. In some embodiments, the additional user interface also includes information related to other applications (e.g., applications different from the health application), devices, and/or software for which the user has provided access to the data used by computer system 600 and/or computer system 624 to determine whether to generate the hypertension notifications.

In some embodiments, computer system 624 is also configured to generate and display notifications in response to a determination that the data collected over the predetermined period of time satisfies the set of notification criteria. For instance, at FIG. 6N, computer system 624 includes display generation component 654 and computer system 624 displays, via display generation component 654, notification 656. Notification 656 can be generated by computer system 624 and/or computer system 600 and can be displayed at the same time as one of notifications 628, 632. As such, the user can be notified about a potential hypertension event on both computer system 600 and computer system 624.

At FIG. 6N, computer system 624 detects user input 650j (e.g., a tap gesture) on notification 656. In response to detecting user input 650j, computer system 624 displays, via display generation component 654, notification user interface 658, as shown at FIG. 6O. Notification user interface 658 includes an indication 658a (e.g., a textual indication) related to the notification and a learn more user interface object 658b. As set forth above, notification 656 and notification user interface 658 do not include a blood pressure measurement (e.g., a numeric and/or quantitative value representing a systolic and/or diastolic blood pressure) of the user. Instead, notification 656 and notification user interface 658 inform the user that the data collected over the predetermined period of time suggests that the user may have hypertension (e.g., the user is at risk of hypertension based on the data collected over the predetermined period of time). In some embodiments, notification user interface 658 includes indication 658a, which may correspond to information region 634a of health event user interface 634. However, notification user interface 658 does not include the information provided in notification details region 636 and device details region 638 of health event user interface 634. As such, indication 658a may prompt the user to use computer system 600 to display health user interface 604 and/or health event user interface 634 to ultimately obtain the information provided in notification details region 636 and device details region 638 of health event user interface 634.

At FIG. 6O, computer system 624 detects user input 650k (e.g., a tap gesture) on learn more user interface object 658b. In response to detecting user input 650k, computer system 624 displays educational user interface 660, as shown at FIG. 6P. Educational user interface 660 provides an indication (e.g., a textual indication) related to information about hypertension, suggested actions for the user to take as a result of receiving notification 656, and/or information about how notification 656 was generated.

FIG. 7 is a flow diagram illustrating a method for generating a notification associated with a health event using an electronic device in accordance with some embodiments. Method 700 is performed at a device (e.g., 100, 300, 500) with one or more output devices and one or more sensors. Some operations in method 700 are, optionally, combined, the orders of some operations are, optionally, changed, and some operations are, optionally, omitted.

As described below, method 700 provides an intuitive way for generating a notification associated with a health event. The method reduces the cognitive burden on a user for accessing information about the health event, thereby creating a more efficient human-machine interface. For battery-operated computing devices, enabling a user to access information about the health event faster and more efficiently conserves power and increases the time between battery charges.

Computer system (e.g., 100, 300, 500, 600, and/or 624) (e.g., an electronic device; a smart device, such as a smart watch or a smart phone; a mobile device; a wearable device) is in communication with one or more output devices (e.g., 602 and/or 654) (e.g., a display generation component (a display, such as a touchscreen display); an audio generation component (e.g., audio circuitry; an integrated speaker; a connected speaker); a haptic output component (e.g., a haptic feedback controller; a haptic motor)) and one or more sensors (e.g., 618, 620, and/or 622) (e.g., a photoplethysmogram sensor, a gyro sensor, and/or an electrocardiogram sensor). In some embodiments, the one or more sensors do not include a sensor for directly and/or specifically measuring a pressure value of a user of the computer system.

Computer system receives (702) first data associated with a user of the computer system from the one or more sensors (e.g., 618, 620, and/or 622) (e.g., periodically and/or intermittently receiving measurement data from the one or more sensors, where the data from the one or more sensors includes data indicative of light absorption of skin of a user associated with the computer system (e.g., one or more measurements received from a photoplethysmogram sensor), data indicative of orientation and/or angular velocity of the computer system (e.g., one or more measurements received from a gyro sensor), and/or data indicative of voltage over time (e.g., one or more measurements of electrical activity of the heart received from an electrocardiogram sensor)). The first data from the one or more sensors (e.g., 618, 620, and/or 622) does not include a direct measurement of pressure (e.g., a value from a sensor that is configured to specifically measure pressure, such as a manometer, a pressure transducer, a strain gauge, and/or a piezometer). In some embodiments, the data from the one or more sensors is data from (e.g., generated by; relating to; about) a user of the computer system. In some embodiments, the data from the one or more sensors includes an indirect measurement of pressure. For instance, raw data from the one or more sensors may be utilized (e.g., as seeds of an algorithm) to calculate and/or estimate pressure, such as systolic blood pressure and/or diastolic blood pressure. In some embodiments, the computer system does not use the data to indirectly determine and/or calculate a numerical measurement of pressure.

In response to receiving the first data from the one or more sensors (704) (e.g., 618, 620, and/or 622) and in accordance with a determination that the first data from the one or more sensors (e.g., 618, 620, and/or 622) satisfies a set of hypertension notification criteria (e.g., the computer system uses the data from the one or more sensors to make an assessment as to whether the user associated with the computer system is at risk for chronic hypertension (e.g., the user associated with the computer system has consistently high blood pressure as opposed to one instance, one estimation, and/or one measurement of blood pressure that is considered high blood pressure); the computer system uses the data from the one or more sensors to generate a risk score indicative of chronic hypertension and the risk score exceeds a threshold value), computer system generates (706) (e.g., outputting; providing; displaying), via the one or more output devices (e.g., 602 and/or 654), a notification (e.g., 628, 632, 656) indicative of a hypertension event (e.g., the data from the one or more sensors indicates that the user associated with the computer system is at risk for chronic hypertension as opposed to one instance (e.g., one measurement and/or one estimation) of high blood pressure) associated with the user of the computer system (e.g., the computer system generates a notification that is displayed by the display generation component of the computer system (e.g., a smart watch) and/or the computer system is in communication with an external device (e.g., a smart phone) and generates a signal received by the external device that causes the external device to display a notification on a display generation component of the external device). In some embodiments, the set of hypertension notification criteria do not include a criterion (e.g., do not include any criterion) based on (e.g., that take into account) a blood pressure value (e.g., diastolic and/or systolic value of a user of the computer system). In some embodiments, the set of hypertension notification criteria do not include a criterion (e.g., any criterion) based on a pressure measurement.

In response to receiving the first data from the one or more sensors (704) (e.g., 618, 620, and/or 622) and in accordance with a determination that the first data from the one or more sensors (e.g., 618, 620, and/or 622) does not satisfy the set of hypertension notification criteria (e.g., the computer system uses the data from the one or more sensors to make an assessment as to whether the user associated with the computer system is at risk for chronic hypertension (e.g., the user associated with the computer system does not have consistently high blood pressure); the computer system uses the data from the one or more sensors to generate a risk score indicative of chronic hypertension and the risk score does not exceed the threshold value), computer system forgoes (708) generating the notification (e.g., 628, 632, 656) of the hypertension event (e.g., a notification is not displayed by the computer system and the external device because the data from the one or more sensors indicates that the user associated with the computer system is not at risk for chronic hypertension).

Conditionally generating a notification indicative of a hypertension event provides the user with feedback regarding the received first data and the relationship of that data to the set of hypertension notification criteria. Providing improved user feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first data associated with a user of the computer system includes data received from the one or more sensors (e.g., 618, 620, and/or 622) over a first period of time that is selected from the group consisting of: a period of greater than 1 hour; a period of greater than 1 day; and a period of greater than 1 week. In some embodiments, the first data is intermittent sensor data from the period of time. In some embodiments, the intermittent data is collected at regular intervals. In some embodiments, the intermittent data is collected at irregular levels. In some embodiments, the first data is continuous sensor data from the period of time.

In some embodiments, the first data associated with a user of the computer system includes a plurality of subsets of data (e.g., the first data is intermittently-collected data) that includes: a first subset of data that is received over a second period of time (e.g., a measurement time window (e.g., 1 second)); and a second subset of data that is received starting after a third period of time (e.g., a time interval between the collection of the first and second subsets of data (e.g., a period of 10 seconds after collecting the first subset of data)) has elapsed after receiving the first subset of data (e.g., after the end of the first period of time). The third period of time is greater than the second period of time.

Conditionally generating a notification based on data that collected over multiple periods of time reduces the risk of transient errors in data collection and decreases the risk of erroneous notifications. Reducing the occurrence of erroneous notifications enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the set of hypertension notification criteria includes a criterion that is satisfied when a risk score (e.g., a quantitative value derived or calculated based on the data) exceeds a threshold value (e.g., an empirically-derived value calculated based a plurality of clinical patients, including patients diagnosed with hypertension and patients that are not diagnosed with hypertension). The risk score is generated based on the first data associated with the user of the computer system.

In some embodiments, the one or more sensors (e.g., 618, 620, and/or 622) are selected from the group consisting of a photoplethysmogram sensor, a gyroscopic sensor, an electrocardiogram sensor, an accelerometer, and a combination thereof.

In some embodiments, the first data associated with the user of the computer system does not include data from a vibration sensor. In some embodiments, the one or more sensors do not include a sensor (e.g., do not include any sensor) that measures vibrations and/or pressure values.

In some embodiments, the computer system is not in communication with (e.g., does not include) a component (e.g., any component) that actively applies mechanical pressure (e.g., a blood pressure cuff) to the user of the computer system. In some embodiments, the one or more sensors (e.g., 618, 620, and/or 622) do not include a sensor (e.g., do not include any sensor) (e.g., a pressure sensor) that measures data within a fourth period of time (e.g., 1 second, 30 seconds, 1 minute, 1 hour) after active, mechanical pressure is applied to the user of the computer system.

In some embodiments, the user of the computer system satisfies a set of feature-eligibility criteria. In some embodiments, the feature-eligibility criteria includes a criterion that is satisfied when the user has been determined to be over a first age (e.g., over 22 years of age). In some embodiments, the method further comprises determining an age of the user (e.g., receiving an input corresponding to the age of the user; receiving data from an external electronic device, data corresponding to the age of the user). In some embodiments, if the user of the computer system does not satisfy the set of feature-eligibility criteria, the notification(s) indicative of a hypertension event are not generated by the computer system. In some embodiments, if the user of the computer system does not satisfy the set of feature-eligibility criteria, no data is collected from the sensors for the purposes of determining whether to provide notification(s) indicative of a hypertension event and no determinations are made based on the set of hypertension notification criteria.

In some embodiments, the set of hypertension notification criteria includes a criterion that is satisfied when the user of the computer system satisfies a set of feature-eligibility criteria. In some embodiments, the feature-eligibility criteria includes a criterion that is satisfied when the user has been determined to be over a first age (e.g., over 22 years of age).

Conditionally generating a notification based on data for users that satisfy a set of feature-eligibility criteria reduces the risk of generating erroneous notifications (e.g., resulting from errors due to the user not being eligible). Reducing the occurrence of erroneous notifications enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, computer system receiving the first data associated with the user of the computer system from the one or more sensors (e.g., 618, 620, and/or 622) includes computer system receiving the first data over a first predetermined time period (e.g., a 1 day period; a 7 day period; a fifteen day period; a 30 day period). In some embodiments, the set of hypertension notification criteria includes a criterion that is satisfied when data has been collected for at least the predetermined time period.

In some embodiments, after computer system receives the first data associated with the user of the computer system from the one or more sensors (e.g., 618, 620, and/or 622) over the first predetermined time period, computer system receives second data associated with the user of the computer system from the one or more sensors (e.g., 618, 620, and/or 622), wherein the second data is received over a second predetermined period of time that does not overlap the first predetermined period of time (e.g., a period of time equal to the first predetermined period of time). In response to computer system receiving the second data from the one or more sensors and in accordance with a determination that the second data from the one or more sensors satisfies the set of hypertension notification criteria, computer system generates, via the one or more output devices (e.g., 602 and/or 654), a second notification (e.g., 632) indicative of a hypertension event associated with the user of the computer system.

In some embodiments, the first data and the second data are non-overlapping sets of data. In some embodiments, a first notification indicative of a hypertension event can be issued (e.g., issued if the first data satisfies the set of hypertension notification criteria) based on the first data and a second notification indicative of a hypertension event can be issued (e.g., issued if the second data satisfies the set of hypertension notification criteria) based on the second data. In some embodiments, the first data is stored in a first data buffer until the first predetermined time period elapses and the computer system resets the first data buffer prior to receiving the second data and storing the second data in the first data buffer.

Conditionally generating first and second notifications based on separate data sets collected makes the first and second notifications independent and reduces the risk that transient, aberrant data will affect multiple notifications. Generating independent notifications and reducing the occurrence of erroneous notifications enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the one or more output devices (e.g., 602 and/or 654) includes a display generation component (e.g., 602 and/or 654) and the computer system is in communication with one or more input devices (e.g., 602 and/or 654) (e.g., a touch-sensitive surface, a mouse, a microphone). Computer system receives a first set of one or more inputs (e.g., 650f), via the one or more input devices (e.g., 602), corresponding to the notification (e.g., 628) (e.g., a touch gesture or mouse click on a displayed notification that is user-selectable graphical object; a verbal command received after an audio notification is outputted). In response to the first set of one or more inputs (e.g., 650f), computer system displays, via the display generation component (e.g., 602), a first user interface (e.g., 634) that includes first information (e.g., 634a, 636a, 636b, 636c, and/or 638) corresponding to the first data (e.g., information on when the data was received; information on the sensor(s); values for the first data and/or subsets of the first data).

Displaying an interface with additional information corresponding to the first data provides the user with feedback regarding the received first data. Providing improved user feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the one or more output devices (e.g., 602 and/or 654) includes a display generation component (e.g., 602 and/or 654). After computer system generates the notification (e.g., 628, 632, and/or 656) indicative of a hypertension event, computer system displays a second user interface (e.g., 644) that includes information (e.g., 646a and/or 646b) (e.g., a time when the notification was generated; a total of notifications that have been generated; data used to generate the notification) corresponding to one or more notifications (e.g., 628, 632, and/or 656) indicative of hypertension events, including the notification (e.g., 628, 632, and/or 656) indicative of a hypertension event. In some embodiments, the second user interface includes a set of one or more user-selectable graphical objects that affect one or more settings associated with notifications indicative of hypertension events (e.g., a frequency of notifications, a duration of notifications, one or more criterion of the set of hypertension notification criteria).

Displaying a second interface with information corresponding to the notification indicative of a hypertension event provides the user with additional feedback about the notification and the data received by the system. Providing improved user feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the notification (e.g., 628, 632, and/or 656) indicative of a hypertension event does not include a numerical (e.g., quantitative) indication of a blood pressure value of a user of the computer system. In some embodiments, the notification does not include any numerical or quantitative values. In some embodiments, the notification only includes non-numerical information.

In some embodiments, the first data associated with a user of the computer system includes a second plurality of subsets of data (e.g., the first data is intermittently-collected data) that includes a third subset of data that is received at a first time and a fourth subset of data that is received a second predetermined period of time after receiving the third subset of data (e.g., at a second time that is a predetermined time). The third subset of data is not received in response to user input (e.g., any user input; the first time is predetermined time; the third subset of data is received independently of user input (e.g., is automatically received)). In some embodiments, the fourth subset of data is not received in response to user input (e.g., any user input; the second time is predetermined time; the fourth subset of data is received independently of user input (e.g., is automatically received)).

Conditionally generating a notification based on separate data sets collected automatically over time reduces the risk of erroneous notifications and of missing the collection of data. Reducing the occurrence of erroneous notifications and reducing the risk of missed data collection enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

Note that details of the processes described above with respect to method 700 (e.g., FIG. 7) are also applicable in an analogous manner to the methods described below. For example, method 900 optionally includes one or more of the characteristics of the various methods described above with reference to method 700. For example, notifications generated via method 700 can be used to prompt a user to initiate a data collection log for a predefined period of time, as set forth in method 900. For brevity, these details are not repeated below.

FIGS. 8A-8U illustrate exemplary user interfaces for managing health information over a predefined period of time, in accordance with some embodiments. The user interfaces in these figures are used to illustrate the processes described below, including the processes in FIG. 9.

FIG. 8A illustrates computer system 600 having display generation component 602 and displaying, via display generation component 602, health user interface 604 of the health application of computer system 600 on a first date (e.g., Monday, May 11th). At FIG. 8A, health user interface 604 includes first health data user interface object 604a and third health data user interface object 604c. First health data user interface object 604a includes information related to a first health parameter (e.g., “Weight”) of a user associated with computer system 600 and third health data user interface object 604c includes information related to a second health parameter (e.g., “Blood Pressure”) of a user associated with computer system 600. In some embodiments, health user interface 604 is a home screen or a default screen of the health application that computer system 600 displays, via display generation component 602, upon launching the health application. In some embodiments, health user interface 604 includes more than two health data user interface objects or one health data user interface object.

At FIG. 8A, computer system 600 detects user input 850a (e.g., a tap gesture) corresponding to selection of third health data user interface object 604c. In response to detecting user input 850a, computer system 600 displays blood pressure user interface 800 on the first date, as shown at FIG. 8B. Blood pressure user interface 800 includes blood pressure graphical element 802 (e.g., a graph) that includes a visual indication (e.g., a set of data points corresponding to systolic pressure and diastolic pressure) of one or more blood pressure measurements received by computer system 600, if available. Computer system 600 is not configured to measure blood pressure (e.g., apply pressure to the user and/or measure a pressure and/or vibration value) of a user associated with the computer system 600. Instead, computer system 600 receives blood pressure data (e.g., blood pressure measurements) via user input and/or via an external device (e.g., a blood pressure measurement device in communication with computer system 600).

Blood pressure data received by computer system 600 is included on blood pressure graphical element 802. At FIG. 8B, blood pressure user interface 800 also includes information user interface object 803. In response to detecting user input corresponding to selection of information user interface object 803, computer system 600 displays an indication of a particular standard and/or rating system associated with the blood pressure data received by computer system 600 and/or displayed on blood pressure graphical element 802. For instance, different countries, regions, and/or areas around the world utilize different rating systems for measuring blood pressure and/or determining whether a person has high, low, and/or normal blood pressure. Accordingly, information user interface object 803 enables computer system 600 to display indicators that provide information as to the particular standard and/or rating system applicable to the blood pressure data corresponding to blood pressure user interface 800.

In some embodiments, computer system 600 filters the blood pressure data displayed on blood pressure graphical element 802. For instance, blood pressure user interface 800 includes date filter 804, which enables computer system 600 to adjust a time scale of blood pressure graphical element 802 and filter blood pressure data displayed on blood pressure graphical element 802 based on date. At FIG. 8B, date filter 804 includes day filter 804a, week filter 804b, month filter 804c, and year filter 804. In some embodiments, date filter 804 may include other suitable periods of time for filtering blood pressure data. At FIG. 8B, day filter 804a of date filter 804 is activated (e.g., selected), and thus, computer system 600 displays all blood pressure data received by computer system 600 for a particular day (e.g., the current day) on blood pressure graphical element 802. In response to detecting user input on week filter 804b, computer system 600 displays all blood pressure data received by computer system 600 for a particular week (e.g., a week that includes the current day) on blood pressure graphical element 802. In response to detecting user input on month filter 804c, computer system 600 displays all blood pressure data received by computer system 600 for a particular month (e.g., a month that includes the current day) on blood pressure graphical element 802. Similarly, in response to detecting user input on year filter 804d, computer system 600 displays all blood pressure data received by computer system for a particular year (e.g., a year that includes the current day) on blood pressure graphical element 802.

In addition to filtering blood pressure data by the time at which it was received by computer system 600 and/or a time stamp associated with the blood pressure data, computer system 600 is also configured to provide indications of particular blood pressure ranges (e.g., normal blood pressure ranges, low blood pressure ranges, and/or high blood pressure ranges) on blood pressure graphical element 802. Thus, a user can visually compare his or her blood pressure data to various blood pressure ranges to better understand his or her health. At FIG. 8B, computer system detects user input 850b (e.g., a tap gesture) corresponding to selection of filter user interface object 806 of blood pressure user interface 800. In response to detecting user input 850b, computer system 600 displays indicators of a normal blood pressure range on blood pressure graphical element 802 on the first date, as shown at FIG. 8C.

At FIG. 8C, blood pressure graphical element 802 includes first indicator 808 and second indicator 810, which represent an upper limit (e.g., first indicator 808) and a lower limit (e.g., second indicator), respectively, for a normal range of a first portion (e.g., systolic pressure) of a blood pressure measurement. In addition, blood pressure graphical element 802 includes third indicator 812 and fourth indicator 814, which represent an upper limit (e.g., third indicator 812) and a lower limit (e.g., fourth indicator 814), respectively, for a normal range of a second portion (e.g., diastolic pressure) of a blood pressure measurement. As such, computer system 600 is configured to display indications of various blood pressure ranges in response to user input on filter user interface object 806, such that the user associated with computer system 600 can easily compare his or her blood pressure measurements to normal blood pressure ranges, low blood pressure ranges, and/or high blood pressure ranges.

At FIG. 8C, blood pressure user interface 800 also includes blood pressure log user interface object 816. As set forth in detail below, in response to user input corresponding to selection of blood pressure log user interface object 816, computer system 600 is configured to activate and/or initiate a schedule for recording blood pressure measurements over a selected time range. In response to activation and/or initiation of the schedule, computer system 600 generates notifications to remind the user to input and/or otherwise provide blood pressure measurements to computer system 600 over the selected time range. As such, the schedule for recording blood pressure measurements facilitates a user's ability to track his or her blood pressure over the selected time range, thereby improving the user's understanding of his or her health.

At FIG. 8C, computer system 600 detects user input 850c (e.g., a tap gesture) corresponding to selection of blood pressure log user interface object 816. In response to detecting user input 850c, computer system 600 initiates a set-up process for activating the schedule for recording blood pressure measurements and displays first set-up user interface 818 on the first date, as shown at FIG. 8D. At FIG. 8D, first set-up user interface 818 includes textual indications that provide the user with information related to the schedule for recording blood pressure measurements. At FIG. 8D, computer system 600 detects user input 850d (e.g., a tap gesture) corresponding to selection of set-up user interface object 818a of first set-up user interface 818. In response to detecting user input 850d, computer system 600 displays second set-up user interface 820 on the first date, as shown at FIG. 8E. Second set-up user interface 820 includes textual indications related to blood pressure measurement devices (e.g., manual blood pressure measurement devices and/or Bluetooth blood pressure measurement devices) that the user can utilize to measure his or her blood pressure. At FIG. 8E, computer system 600 detects user input 850e (e.g., a tap gesture) corresponding to selection of next user interface object 820a of second set-up user interface 820. In response to detecting user input 850e, computer system 600 displays third set-up user interface 822 on the first date, as shown at FIG. 8F.

At FIG. 8F, third set-up user interface 822 includes first time range user interface object 822a, second time range user interface object 822b, and next user interface object 822c. Computer system 600 can initiate the schedule for recording blood pressure measurements over different time ranges based on user selection of first time range user interface object 822a or second time range user interface object 822b. At FIG. 8F, first time range user interface object 822a corresponds to a first time range of seven days and second time range user interface object 822b corresponds to a second time range of 30 days. As set forth below, the schedule for recording blood pressure measurements over the first time range can include a first number of number of blood pressure measurements, such as two blood pressure measurements per day over the seven day range. The schedule for recording blood pressure measurements over the second time range can include a second number of blood pressure measurements, different from the first number of blood pressure measurements, such as one blood pressure measurement per day over the 30 day range. As such, computer system 600 determines different periods of time (e.g., half of one day or one full day) within the selected time range for recording respective blood pressure measurements based on which time range is selected.

At FIG. 8F, computer system 600 detects user input 850f (e.g., a tap gesture) corresponding to selection of first time range user interface object 822a. In some embodiments, in response to detecting user input 850f, computer system 600 displays indicator 824 on first time range user interface object 822a indicating that the first time range associated with first time range user interface object 822a is selected. After detecting user input 850f, computer system 600 detects user input 850g (e.g., a tap gesture) corresponding to selection of next user interface object 822c. After detecting user input 850g (and, optionally, after detecting user inputs 850i, 850j, and/or 850k described below with reference to FIGS. 8G-8I), computer system 600 displays blood pressure user interface 800, as shown at FIG. 8J.

Alternatively, at FIG. 8F, computer system 600 detects user input 850h (e.g., a tap gesture) corresponding to selection of second time range user interface object 822b. In some embodiments, in response to detecting user input 850h, computer system 600 displays indicator 824 on second time range user interface object 822b (e.g., instead of on first time range user interface object 822a) indicating that the second time range associated with second time range user interface object 822b is selected. After detecting user input 850h, computer system detects user input 850g corresponding to selection of next user interface object 822c. After detecting user input 850g (and, optionally, after detecting user inputs 850i, 850j, and/or 850k described below with reference to FIGS. 8G-8I), computer system 600 displays blood pressure user interface 800, as shown at FIG. 8K. In some embodiments, computer system 600 deactivates and/or forgoes display of next user interface object 822c before computer system 600 detects user input 850f or user input 850h. Accordingly, computer system 600 does not process and/or detect user input 850g until after receiving user input corresponding to selection of one of first time range user interface object 822a or second time range user interface object 822b.

While FIG. 8F shows selection of a time range for the schedule for recording blood pressure measurements as user interface objects 822a and 822b, in some embodiments, third set-up user interface 822 includes, in addition to user interface objects 822a and 822b and/or in lieu of user interface objects 822a and 822b, a user interface object enabling a user to enter a custom time range via a keyboard or other input device.

At FIG. 8F, in response to detecting user input 850g, computer system 600 displays fourth set-up user interface 826 on the first date, as shown at FIG. 8G. Fourth set-up user interface 826 includes textual indications that inform the user that computer system 600 generates notifications reminding the user to input blood pressure measurements in accordance with the schedule and/or the selected time range. In some embodiments, fourth set-up user interface 826 includes one or more user interface objects that, when selected, cause computer system 600 to deactivate notification reminders associated with the schedule for recording blood pressure measurements. At FIG. 8G, computer system 600 detects user input 850i (e.g., a tap gesture) corresponding to selection of next user interface object 826a of fourth set-up user interface 826. In response to detecting user input 850i, computer system 600 displays fifth set-up user interface 828 on the first date, as shown at FIG. 8H. Fifth set-up user interface 828 includes textual indications that provide information to the user on how to measure blood pressure to obtain a blood pressure measurement that is ultimately provided to computer system 600 (e.g., via user input and/or transmission from an external device). At FIG. 8H, computer system 600 detects user input 850j (e.g., a tap gesture) corresponding to selection of next user interface object 828a of fifth set-up user interface 828. In response to detecting user input 850j, computer system 600 displays sixth set-up user interface 830 on the first date, as shown at FIG. 8I.

Sixth set-up user interface 830 includes log blood pressure measurement user interface object 830a and skip blood pressure measurement user interface object 830b. As such, sixth set-up user interface 830 enables a user to provide a first blood pressure measurement for the schedule (e.g., via selection of log blood pressure measurement user interface object 830a) and/or complete the set-up and provide the first blood pressure measurement for the schedule at a time after the current time (e.g., via selection of skip blood pressure measurement user interface object 830b). At FIG. 8I, computer system 600 detects user input 850k (e.g., a tap gesture) corresponding to selection of skip blood pressure measurement user interface object 830b. In response to detecting user input 850k, computer system 600 displays blood pressure user interface 800 on the first date, as shown at FIGS. 8J and 8K. As set forth below, computer system 600 displays blood pressure user interface 800 including first schedule user interface object 832 (FIG. 8J) corresponding to the first time range or second schedule user interface object 834 (FIG. 8K) corresponding to the second time range based on computer system 600 detecting user input 850f or 850h at FIG. 8F.

Alternatively, at FIG. 8I, computer system 600 detects user input 850l (e.g., a tap gesture) corresponding to selection of log blood pressure measurement user interface object 830a. In response to detecting user input 850l, computer system 800 displays measurement input user interface 854, as shown at FIG. 8O.

Turning now to FIG. 8J, computer system 600 detects user input 850f corresponding to selection of first time range user interface object 822a and displays blood pressure user interface 800 having first schedule user interface object 832 on the first date. First schedule user interface object 832 corresponds to the schedule being over the first time range of seven days. Accordingly, first schedule user interface object 832 includes day indicators 832a-832g corresponding to respective days of a week. In addition, first schedule user interface object 832 includes first interval indicator 832h and second interval indicator 832i. Because the first time range associated with first schedule user interface object 832 is for a relatively short period of time (i.e., seven days versus 30 days), first schedule user interface object 832 provides for multiple blood pressure measurements per day. At FIG. 8J, first schedule user interface object 832 includes measurement indicators 836 representing time periods (e.g., intervals) within the first time range for respective blood pressure measurements. In other words, measurement indicators 836 indicate various sub-ranges of time (e.g., a half of a day or 12 hours) within the first time range that computer system 600 is configured to prompt the user to provide (e.g., input and/or otherwise transmit) a blood pressure measurement to computer system 600.

In addition, computer system 600 displays add measurement user interface object 838 on blood pressure user interface 800. In response to detecting user input corresponding to selection of add measurement user interface object 838, computer system 600 displays measurement input user interface 854, as shown at FIG. 8O. As set forth in detail below with reference to FIG. 8O, a blood pressure measurement received by computer system 600 via measurement input user interface 854 corresponds to a respective measurement indicator 836 that is associated with a current day and/or a current time of day. In other words, in response to detecting user input corresponding to selection of add measurement user interface object 838, computer system 600 is configured to receive and record a blood pressure measurement that corresponds to the respective measurement indicator 836 associated with the current day and/or the current time of day without receiving and recording a blood pressure measurements that corresponds to a measurement indicator 836 that is not associated with the current day and/or the current time of day.

As set forth above, in response to detecting user input 850h corresponding to selection of second time range user interface object 822b, computer system 600 displays blood pressure user interface 800 having second schedule user interface object 834, as shown at FIG. 8K. Second schedule user interface object 834 corresponds to the schedule being over the second time range of 30 days. Accordingly, second schedule user interface object 834 includes day indicators 834a-834g corresponding to respective days of a week for five weeks (e.g., rows within second schedule user interface object 834) that occur over the course of 30 days. In contrast to first schedule user interface object 832, second schedule user interface object 834 does not include interval indicators. Because the second time range associated with second schedule user interface object 834 is for a relatively long period of time (i.e., 30 days versus seven days), second schedule user interface object 834 provides for a single blood pressure measurement per day. In other words, because the second time range includes several weeks, the schedule for recording blood pressure measurements receives a sufficient amount of data to identify trends and/or patterns in the user's blood pressure even when the data is entered and/or received once per day.

At FIG. 8K, second schedule user interface object 834 includes measurement indicators 840 representing each day within the second time range for respective blood pressure measurements. In other words, measurement indicators 840 indicate various sub-ranges of time (e.g., a single day or 24 hours) within the second time range that computer system 600 is configured to prompt the user to provide (e.g., input and/or otherwise transmit) a blood pressure measurement to computer system 600.

At FIG. 8K, computer system 600 detects user input 850m (e.g., a tap gesture) corresponding to selection of add measurement user interface object 838. In response to detecting user input 850m, computer system 600 displays measurement input user interface 854, as shown at FIG. 8O. As set forth in detail below with reference to FIG. 8O, a blood pressure measurement received by computer system 600 via measurement input user interface 854 corresponds to a respective measurement indicator 840 that is associated with a current day and/or a current time of day. In other words, in response to detecting user input corresponding to selection of add measurement user interface object 838, computer system 600 is configured to receive and record a blood pressure measurement that corresponds to the respective measurement indicator 840 associated with the current day and/or the current time of day without recording a blood pressure measurement that corresponds to a measurement indicator 840 that is not associated with the current day and/or the current time of day.

Turning now to FIG. 8L, computer system 600 displays notification 842 on user interface 844 (e.g., a lock screen user interface and/or a home screen user interface) on a second date (e.g., Friday, May 15th) after the first date. Notification 842 corresponds to a confirmation that a blood pressure measurement was received within a time period of the first time range and/or the second time range corresponding to the current day and/or the current time of day. For example, notification 842 provides a confirmation to the user that a blood pressure measurement was received for the morning of Friday, May 15th when the time range is the first time range (e.g., seven days) and/or that the blood pressure measurement was received for the day of Friday, May 15th when the time range is the second time range (e.g., 30 days). In some embodiments, computer system 600 displays notification 842 after receiving a blood pressure measurement (e.g., via user input while computer system 600 displays measurement input user interface 854). In some embodiments, computer system 600 displays notification 842 in response to receiving a blood pressure measurement from an external device (e.g., a blood pressure measurement device configured to transmit a wireless signal indicative of the blood pressure measurement to computer system 600 via Bluetooth, Wi-Fi, and/or Zigbee). In some embodiments, computer system 600 displays notification 842 in response to receiving any blood pressure measurement before computer system 600 displays a reminder notification (e.g., notification 846 at FIG. 8M) prompting the user to provide a blood pressure measurement for the period of time within the first time range and/or the second time range corresponding to the current day and/or current time of day.

Computer system 600 is configured to prompt the user to provide a blood pressure measurement for the period of time within the first time range and/or the second time range corresponding to the current day and/or current time of day in response to detecting that a blood pressure measurement has not been received. At FIG. 8M, computer system 600 displays notification 846 on user interface 844 on the second date in response to detecting that a blood pressure measurement has not been received within a threshold time (e.g., 1 hour, 6 hours, 8 hours, 10 hours, 12 hours) from the beginning of the period of time (e.g., 12 am of a day and/or 12 μm of a day) of the first time range and/or the second time range corresponding to the current day and/or the current time of day. Accordingly, computer system 600 prompts and/or reminds the user to provide the blood pressure measurement so that computer system 600 can receive a blood pressure measurement for each period of time within the first range and/or the second time range.

In some embodiments, in response to receiving a blood pressure measurement (e.g., via an external device, such as a Bluetooth enabled blood pressure device and/or sensor), computer system 600 is configured to remove and/or cease displaying notification 846 on user interface 844 and/or from another user interface associated with the health application. Accordingly, computer system 600 clears reminder notifications when the reminder to provide a blood pressure measurement is no longer appropriate.

At FIG. 8M, computer system 600 detects user input 850n (e.g., a long press gesture) corresponding to notification 846. In response to detecting user input 850n, computer system 600 displays skip blood pressure measurement user interface object 848 and log blood pressure measurement user interface object 852 on user interface 844 on the second date, as shown at FIG. 8N. In response to receiving user input corresponding to selection of skip blood pressure measurement user interface object 848, computer system 600 clears and/or removes notification 846 from user interface 844. In some embodiments, computer system 600 provides an additional notification reminding user to provide a blood pressure measurement after a predetermined period of time from detecting user input corresponding to selection of skip blood pressure measurement user interface object 848.

At FIG. 8N, computer system 600 detects user input 850o (e.g., a tap gesture) corresponding to selection of log blood pressure measurement user interface object 852. In response to detecting user input 850o, computer system 600 displays measurement input user interface 854 on the second date, as shown at FIG. 8O. As set forth above, computer system 600 can also display measurement input user interface 854 in response to user input 850l and/or 850m.

Measurement input user interface 854 includes first measurement user interface object 854a and second measurement user interface object 854b. At FIG. 8O, computer system 600 detects user input 850p (e.g., a tap gesture) corresponding to selection of first measurement user interface object 854a. In addition, computer system 600 detects one or more user inputs (e.g., via a keyboard and/or other input device) that correspond to a numeric value of a first measurement of blood pressure, such as a systolic pressure from a blood pressure measurement. At FIG. 8O, computer system 600 also detects user input 850q (e.g., a tap gesture) corresponding to selection of second measurement user interface object 854b. In addition, computer system 600 detects one or more user inputs (e.g., via a keyboard and/or other input device) that correspond to a numeric value of a second measurement of blood pressure, such as a diastolic pressure from a blood pressure measurement. In some embodiments, measurement input user interface 854 includes a single measurement user interface object that enables a user to input both portions (e.g., systolic and diastolic pressure) of a blood pressure measurement.

At FIG. 8O, measurement input user interface 854 is displayed at 9:41 PM on May 15th (e.g., the current day). As such, inputs corresponding to the first measurement user interface object 854a and the second measurement user interface object 854b (e.g., collectively a blood pressure measurement) correspond to a respective measurement indicator 836 and/or 840 for the evening of and/or the entire day of Friday, May 15th. At FIG. 8O, measurement input user interface 854 does not include a user interface object that enables computer system 600 to receive a measurement corresponding to another measurement indicator that is not associated with the current day and/or the current time of day.

In some embodiments, computer system 624, which is in communication with computer system 600 (e.g., and associated with the same user as computer system 600), is also configured to provide notifications related to the schedule for collecting blood pressure measurements. For instance, at FIG. 8P, computer system 624 displays notification user interface 856. Computer system 624 displays notification user interface 856 in response to a determination that computer system 600 has not received a blood pressure measurement for the period of time within the first time range and/or the second time range. In some embodiments, computer system 624 displays notification user interface 856 when computer system 600 displays notification 846.

At FIG. 8P, notification user interface 856 includes first measurement user interface object 856a and second measurement user interface object 856b. At FIG. 8P, computer system 624 detects user input 850r (e.g., a tap gesture) corresponding to selection of first measurement user interface object 856a. In addition, computer system 624 detects one or more user inputs (e.g., via a keyboard and/or another input device) that correspond to a numeric value of a first measurement of blood pressure, such as a systolic pressure from a blood pressure measurement. At FIG. 8P, computer system 624 also detects user input 850s (e.g., a tap gesture) corresponding to selection of second measurement user interface object 856b. In addition, computer system 624 detects one or more user inputs (e.g., via a keyboard and/or another input device) that correspond to a numeric value of a second measurement of blood pressure, such as a diastolic pressure from a blood pressure measurement. In some embodiments, notification user interface 856 includes a single measurement user interface object that enables a user to input both portions (e.g., systolic and diastolic pressure) of a blood pressure measurement.

At FIG. 8P, notification user interface 856 is displayed at 9:41 PM on May 15th (e.g., the current day). As such, inputs corresponding to the first measurement user interface object 856a and the second measurement user interface object 856b (e.g., collectively a blood pressure measurement) correspond to a respective measurement indicator 836 and/or 840 for the evening of and/or the entire day of Friday, May 15th. At FIG. 8P, measurement input user interface 856 does not include a user interface object that enables computer system 624 to receive a measurement corresponding to another measurement indicator that is not associated with the current day and/or the current time of day.

In response to receiving a blood pressure measurement via measurement input user interface 854 and/or notification user interface 856, computer system 600 updates a respective measurement indicator 836 and/or 840 to reflect that the blood pressure measurement is received, as shown at FIG. 8Q. At FIG. 8Q, computer system 600 displays blood pressure user interface 800, which includes second schedule user interface object 834 corresponding to the second time range. In response to receiving the blood pressure measurement for the second date (e.g., Friday, May 15th), computer system 600 updates an appearance of measurement indicator 840a (e.g., fills measurement indicator 840a with a check mark) corresponding to the second date (e.g., Friday, May 15th) to indicate that computer system 600 received the blood pressure measurement corresponding to the time period associated with measurement indicator 840a. In addition, computer system 600 updates blood pressure graphical element 802 to include an indication (e.g., a pair of data points) corresponding to the received blood pressure measurement for the second date. In some embodiments, computer system 600 ceases to display add measurement user interface object 838 and/or adjusts an appearance of add measurement user interface object 838 to further indicate that a blood pressure measurement has been received for the current time period associated with the current day and/or the current time of day.

At FIG. 8Q, computer system 600 has also updated the appearance of measurement indicators 840b, 840d, and 840e indicating that computer system 600 also received the blood pressure measurements for the days associated with measurement indicators 840b, 840d, and 840e. In addition, computer system 600 has not updated the appearance of measurement indicator 840c, corresponding to Tuesday, May 12th, indicating that computer system 600 did not receive a blood pressure measurement for that day that is within the second range.

In some embodiments, computer system 600 is configured to receive blood pressure measurements for days and/or other periods of time (e.g., half of days) that occur outside of the current period of time (e.g., the current day and/or the current time of day). At FIG. 8Q, computer system 800 receives user input 850t corresponding to add data user interface object 858 of blood pressure user interface 800. In response to detecting user input 850t, computer system 600 displays previous measurement input user interface 860, as shown at FIG. 8R. Previous measurement input user interface 860 is similar to measurement input user interface 854, but also includes date user interface object 860a and time user interface object 860b. In response to detecting one or more user inputs corresponding to date user interface object 860a, computer system 600 is configured to update a date indicator 860c displayed in date user interface object 860a. Date indicator 860c corresponds to a date on which an input blood pressure measurement was taken (e.g., a blood pressure measurement that is input via first measurement user interface object 854a and second measurement user interface object 854b). Similarly, in response to detecting one or more user inputs corresponding to time user interface object 860b, computer system 600 is configured to update a time indicator 860d displayed in time user interface object 860b. Time indicator 860d corresponds to a time on the date associated with date indicator 860c at which the input blood pressure measurement taken (e.g., the blood pressure measurement that is input via first measurement user interface object 854a and second measurement user interface object 854b). As such, in some embodiments, computer system 600 is configured to receive blood pressure measurements that are associated with a day and/or a time that is prior to or before a current day (e.g., a period of time within the first time range and/or the second time range that is not associated with the current day and/or the current time of day).

Turning now to FIG. 8S, computer system 600 displays blood pressure user interface 800 having second schedule user interface object 834 corresponding to the second time range on a third date (e.g., Tuesday, June 9th). At FIG. 8S, computer system 600 has updated each of measurement indicators 840 of second schedule user interface object 834, thereby indicating that a blood pressure measurement has been received for each period of time (e.g., each day) of the second time range (e.g., 30 days). In other words, at FIG. 8S, computer system 600 displays blood pressure user interface 800 having second schedule user interface 834 after completion of the second time range (e.g., 30 days has elapsed from the time the schedule for collecting blood pressure measurements was initiated). In response to detecting completion of the second time range, computer system 600 further displays share user interface object 862 and new schedule user interface object 864. Therefore, in some embodiments, computer system 600 forgoes display of share user interface object 862 and/or new schedule user interface object 864 until the second time range (and/or another selected time range) has elapsed.

Share user interface object 862 enables the received blood pressure measurements during the second time range to be shared with an external device. For example, in response to user input corresponding to selection of share user interface object 862, computer system 600 generates an exportable file that includes the received blood pressure measurements during the second time range. Accordingly, computer system 600 can transmit the exportable file to an external device via electronic mail, via short messaging service, via Bluetooth, via Wi-Fi, via short range communication, and/or via another suitable communication technique.

New schedule user interface object 864 enables computer system 600 to begin a new schedule for collecting blood pressure measurements. For example, in response to detecting user input corresponding to selection of new schedule user interface object 864, computer system 600 displays first set-up user interface 818 and/or third set-up user interface 822. As such, computer system 600 is configured to receive one or more inputs that cause computer system to activate and/or initiate another schedule for collecting blood pressure measurements.

As set forth above, computer system 600 is configured to generate hypertension notifications in response to a determination that a set of notification criteria (e.g., hypertension notification criteria) has been satisfied. At FIG. 8T, computer system 600 displays notification 628 on user interface 630 (e.g., a lock screen user interface and/or a home screen user interface). At FIG. 8T, computer system 600 detects user input 850u (e.g., a long press gesture) on notification 628. In response to detecting user input 850u, computer system 600 displays new schedule user interface object 866 and/or skip schedule user interface object 868 on user interface 630, as shown at FIG. 8U. Accordingly, computer system 600 can activate and/or initiate a new schedule for collecting blood pressure measurements in response to user input 850u on notification 628 and an additional user input corresponding to selection of new schedule user interface object 866. Therefore, a user can cause computer system 600 to activate and/or initiate the schedule for collecting blood pressure measurements after computer system 600 generates a hypertension notification. In contrast, in response to detecting user input corresponding to skip schedule user interface object 868, computer system 600 clears and/or removes notification 628 from user interface 630.

FIG. 9 is a flow diagram illustrating a method for managing health information over a predefined time range using an electronic device in accordance with some embodiments. Method 900 is performed at a device (e.g., 100, 300, 500, 600, and/or 624) with a display generation component and one or more input devices. Some operations in method 900 are, optionally, combined, the orders of some operations are, optionally, changed, and some operations are, optionally, omitted.

As described below, method 900 provides an intuitive way for managing health information over a predefined time range. The method reduces the cognitive burden on a user for tracking and/or monitoring health information, thereby creating a more efficient human-machine interface. For battery-operated computing devices, enabling a user to track and/or monitor health information faster and more efficiently conserves power and increases the time between battery charges.

Computer system (e.g., 100, 300, 500, 600, and/or 624) (e.g., an electronic device; a smart device, such as a smart watch or a smart phone; a mobile device; a wearable device) is in communication with a display generation component (e.g., 602 and/or 654) (e.g., a display, such as a touchscreen display) and one or more input devices (e.g., 602 and/or 654) (e.g., a keyboard, a mouse, a touch-sensitive surface).

Computer system receives (902), via the one or more input devices (e.g., 602 and/or 654), a first set of one or more inputs (e.g., 850a, 850c, 850d, 850e, 850f, 850g, 850h, 850i, 850j, and/or 850k) (e.g., one or more taps, gestures, mouse clicks) that includes a first input (e.g., 850f and/or 850h) selecting a time range (e.g., the selection of a user-interactive graphical user interface object that corresponds to a first time range from among a plurality of user-interactive graphical user interface objects corresponding to different time ranges; the entry of values corresponding to a time range).

In response to receiving the first set of one or more inputs, computer system displays (904), via the display generation component (e.g., 602 and/or 654), a first user interface (e.g., 800) (e.g., a user interface including data and/or information associated with recorded measurements of a user characteristic (e.g., blood pressure)).

In accordance with a determination that the first user input (e.g., 850f) corresponds to selection of a first time range (e.g., a week; two weeks; a month), the first user interface (e.g., 800) includes (906) a first plurality of measurement indicators (e.g., 836) (e.g., 14 measurement indicators) within the first time range.

In accordance with a determination that the first user input (e.g., 850h) corresponds to selection of a second time range (e.g., a week; two weeks; a month), different from the first time range, the first user interface (e.g., 800) includes (908) a second plurality of measurement indicators (e.g., 840) (e.g., 30 measurement indicators) within the second time range. In some embodiments, the first time range is a subset of the second time range (e.g., the first time range is a first week of the month and the second time range is the entire month).

The first user interface (e.g., 800) also includes (910) a first measurement entry user-interactive graphical user interface object (e.g., 838) (e.g., an add measurement affordance corresponding to a respective measurement indicator based on the first user input) that, when selected via the one or more input devices (e.g., 602) (e.g., via a tap gesture on a touch-sensitive surface), initiates a measurement-entry process (e.g., displaying a second user interface that includes one or more user-interactive graphical user interface objects enabling a user to enter a blood pressure measurement).

In accordance with the first user interface (e.g., 800) including the first plurality of measurement indicators (e.g., 836) within the first time range, the measurement entry process includes (912) computer system entering a blood pressure measurement (e.g., a systolic and/or diastolic measurement value) that corresponds to the first time range and a current day (e.g., the current day and/or the current time of day when the first measurement entry user-interactive graphical user interface object is selected). In some embodiments, the blood pressure measurement corresponds to the first time and range and corresponds to a specific measurement indicator in the first plurality of measurement indicators (e.g., a specific measurement indicator in the first plurality of measurement indicators corresponding to a current day and/or a current time of day).

In accordance with the first user interface (e.g., 800) including the second plurality of measurement indicators (e.g., 840) within the second time range, the measurement entry process includes (914) computer system entering a blood pressure measurement that corresponds to the second time range and the current day (e.g., the current day and/or the current time of day when the first measurement entry user-interactive graphical user interface object is selected). In some embodiments, the blood pressure measurement corresponds to the second time range and corresponds to a specific measurement indicator in the second plurality of measurement indicators (e.g., a specific measurement indicator in the second plurality of measurement indicators corresponding to a current day and/or a current time of day).

Displaying a user interface that includes either a first plurality of measurement indicators or a second plurality of measurement indicators depending on a selected time range provides the user with control over contents of the user interface that is used for subsequent inputs relating to blood pressure measurements, thereby improving the user-system interaction. Improving the user-system interaction enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first plurality of measurement indicators (e.g., 836) (e.g., 14) includes a first number of measurement indicators and the second plurality of measurement indicators (e.g., 840) includes a second number of measurement indicators (e.g., 30), different from the first number. In some embodiments, the first plurality of measurement indicators includes multiple indicators for a single, first day and the second plurality of measurement indicators includes a single indicator for the single, first day.

Displaying a different number of measurement indicators depending on a selected time range provides the user with control over contents of the user interface that is used for subsequent inputs relating to blood pressure measurements, thereby improving the user-system interaction. Improving the user-system interaction enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the computer system is in communication with one or more blood pressure sensors (e.g., integrated sensors; sensors of an external blood pressure measurement device that are in wireless communication with the computer system). Computer system receives (e.g., wirelessly), via the one or more blood pressure sensors, first data corresponding to a first blood pressure measurement, wherein the first data is not received in response to user input (e.g., any user input) at the computer system (e.g., the first data is automatically received).

In some embodiments, the first data is received while a first notification (e.g., 846) is active (e.g., flagged for display in a notification center; marked as unread; is actively displayed on a display of the computer system). In response to receiving the first data, computer system deactivates the first notification (e.g., 846) (e.g., ceases to display the displayed notification; marking the notification as read; de-flagging the notification).

Automatically deactivating a notification when the first data is received assists the user with the management of notifications. Performing an operation automatically enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, in response to receiving the first data, computer system displays, via the display generation component (e.g., 602), an indication (e.g., 842) (e.g., a notification) that the first data was received. In some embodiments, the notification includes an indication of a time period of the current day that the first data has been associated with (e.g., the entire day, the morning, the evening).

Displaying an indication in response to receiving the first data provides the user with feedback as to the data that was received. Providing improved user feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first plurality of measurement indicators (e.g., 836) includes a first measurement indicator that represents (e.g., corresponds to) a first time period (e.g., 12 hours) and the second plurality of measurement indicators (e.g., 840) includes a second measurement indicator (e.g., 840a, 840b, 840c, 840d, and/or 840e) that represents (e.g., corresponds to) a second time period (e.g., 1 day), different from the first time period.

In some embodiments, the first set of one or more inputs (e.g., 850a, 850c, 850d, 850e, 850f, 850g, 850h, 850i, 850j, and/or 850k) includes a first input (e.g., 850f and/or 850h) (e.g., a tap gesture; a mouse click) corresponding to a first user-selectable graphical user interface object (e.g., 822a) that corresponds to the first time range or to a second user-selectable graphical user interface object (e.g., 822b) that corresponds to the second time range. In some embodiments, the first and second user-selectable graphical user interface objects are displayed on the same user interface.

Providing selectable graphical user interface objects for selecting a time range provides the user with feedback as to whether the first or second plurality of measurement indicators will be displayed in response to the input. Providing improved user feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, after computer system receives the first set of one or more inputs (e.g., 850a, 850c, 850d, 850e, 850f, 850g, 850h, 850i, 850j, and/or 850k) (e.g., after displaying the first user interface) and in accordance with a determination that predetermined period of time (e.g., 1 minute; 5 minutes; 30 minutes, 60 minutes; 12 hours; 24 hours) has elapsed (e.g., has passed) since a first event (e.g., since a last blood pressure measurement was received; since a first time (e.g., since 8 AM; since 5 PM); since the first set of one or more inputs were received) without having received (e.g., automatically received; manually received) a blood pressure measurement, computer system displays, via the display generation component (e.g., 602 and/or 654), a reminder notification (e.g., 846 and/or 856) (e.g., a notification that indicates that a blood pressure measurement has not been received).

Automatically displaying a reminder notification when certain conditions are met performs the function without requiring further user input and provides the user with feedback about the condition being met. Performing an operation automatically and providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, in accordance with the first user interface (e.g., 800) including the first plurality of measurement indicators (e.g., 836) within the first time range and after (e.g., in response to) computer system enters a blood pressure measurement that corresponds to the first time range, computer system modifies the appearance (e.g., marking as completed; changing a color; changing a shape) of a first measurement indicator (e.g., an indicator that corresponds to the first half of the current day) of the first plurality of measurement indicators (e.g., 836). In accordance with the first user interface (e.g., 800) including the second plurality of measurement indicators (e.g., 840) within the second time range and after (e.g., in response to) computer system enters a blood pressure measurement that corresponds to the second time range, computer system modifies the appearance of a second measurement indicator (e.g., 840a) (e.g., an indicator that corresponds to the entirety of the current day) of the second plurality of measurement indicators (e.g., 840).

Modifying the appearance of a respective measurement indicator in response to receiving a blood pressure measurement provides the user with feedback as to which indicator the measurement has been associated with. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first user interface (e.g., 800) includes a graphical depiction (e.g., 802) (e.g., a graph (e.g., a line graph; a scatter plot graph); a table) of one or more received blood pressure measurements.

Displaying a graphical depiction of received measurement data provides the user with feedback as blood pressure measurements that have been received. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first user interface (e.g., 800) includes a third user-selectable graphical object (e.g., 803) that, when selected, causes display (e.g., in the first user interface) of information (e.g., a scale; a standard shown in the depiction) corresponding to the graphical depiction (e.g., 802) (e.g., to a parameter or aspect of the depiction).

Displaying information regarding the graphical depiction provides the user with feedback as to an aspect of the graphical depiction and of the blood measurements depicted in the depiction. Providing improved feedback enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the first user interface (e.g., 800) includes a fourth user-selectable graphical object (e.g., 858) that, when selected, causes computer system to initiate a process (e.g., displaying a second user interface that includes one or more user-interactive graphical user interface objects enabling a user to enter a blood pressure measurement) for receiving (e.g., via manual entry) a blood pressure measurement (e.g., a systolic and/or diastolic measurement value) for a time period of a day that precedes the current day (e.g., a past day).

Providing an object for entering measurements for previous time periods provides the user with additional control options, thereby promoting improved and sustained user-system interactions. Improving and sustaining user-system interactions enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments the first user interface (e.g., 800) includes a fifth user-selectable graphical object (e.g., 862) that, when selected, causes computer system to initiate a process for transmitting data corresponding to one or more received blood pressure measurements to an external electronic device (e.g., a computer associated with a medical provider).

In some embodiments, the first user interface (e.g., 800) includes a sixth user-selectable graphical object (e.g., 864) (e.g., a new journal period affordance) that, when selected, causes computer system to initiate a process for selecting a second time range (e.g., the selection of a user-interactive graphical user interface object that corresponds to a first time range from among a plurality of user-interactive graphical user interface objects corresponding to different time ranges; the entry of values corresponding to a time range) and for displaying a second user interface (e.g., 800) (e.g., that includes one or more features of the first user interface) that includes a third plurality of measurement indicators (e.g., 836 and/or 840) that includes a number of measurement indicators selected based on the second time range. In some embodiments, the sixth affordance is included (only included; first included) in the first user interface in accordance with a determination that a sufficient number (e.g., 14; 30) of blood pressure measurements have been received and/or a determination that a predetermined period of time (e.g., 7 days; 30 days) has elapsed.

Providing an object for initiating a process to select a new time range provides the user with additional control options, thereby promoting improved and sustained user-system interactions. Improving and sustaining user-system interactions enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

In some embodiments, the computer system is in communication with one or more sensors (e.g., 618, 620, and/or 622) (e.g., a photoplethysmogram sensor, a gyro sensor, and/or an electrocardiogram sensor). In some embodiments, the one or more sensors do not include a sensor for directly and/or specifically measuring a pressure value of a user of the computer system. Computer system receives (702) first sensor data associated with a user of the computer system from the one or more sensors (e.g., 618, 620, and/or 622) (e.g., periodically and/or intermittently receiving measurement data from the one or more sensors, where the data from the one or more sensors includes data indicative of light absorption of skin of a user associated with the computer system (e.g., one or more measurements received from a photoplethysmogram sensor), data indicative of orientation and/or angular velocity of the computer system (e.g., one or more measurements received from a gyro sensor), and/or data indicative of voltage over time (e.g., one or more measurements of electrical activity of the heart received from an electrocardiogram sensor)). In some embodiments, the first sensor data from the one or more sensors does not include a direct measurement of pressure (e.g., a value from a sensor that is configured to specifically measure pressure, such as a manometer, a pressure transducer, a strain gauge, and/or a piezometer). In some embodiments, the data from the one or more sensors is data from (e.g., generated by; relating to; about) a user of the computer system. In some embodiments, the data from the one or more sensors includes an indirect measurement of pressure. For instance, raw data from the one or more sensors may be utilized (e.g., as seeds of an algorithm) to calculate and/or estimate pressure, such as systolic blood pressure and/or diastolic blood pressure. In some embodiments, the computer system does not use the data to indirectly determine and/or calculate a numerical measurement of pressure.

In response to receiving the first sensor data from the one or more sensors (704) (e.g., 618, 620, and/or 622) and in accordance with a determination that the first sensor data from the one or more sensors (e.g., 618, 620, and/or 622) satisfies a set of hypertension notification criteria (e.g., the computer system uses the data from the one or more sensors to make an assessment as to whether the user associated with the computer system is at risk for chronic hypertension (e.g., the user associated with the computer system has consistently high blood pressure as opposed to one instance, one estimation, and/or one measurement of blood pressure that is considered high blood pressure); the computer system uses the data from the one or more sensors to generate a risk score indicative of chronic hypertension and the risk score exceeds a threshold value), computer system displays, via the display generation component (e.g., 602), a notification (e.g., 628) indicative of a hypertension event (e.g., the data from the one or more sensors indicates that the user associated with the computer system is at risk for chronic hypertension as opposed to one instance (e.g., one measurement and/or one estimation) of high blood pressure) associated with the user of the computer system (e.g., the computer system generates a notification that is displayed by the display generation component of the computer system (e.g., a smart watch) and/or the computer system is in communication with an external device (e.g., a smart phone) and generates a signal received by the external device that causes the external device to display a notification on a display generation component of the external device). In some embodiments, the set of hypertension notification criteria do not include a criterion (e.g., do not include any criterion) based on (e.g., that take into account) a blood pressure value (e.g., diastolic and/or systolic value of a user of the computer system). In some embodiments, the set of hypertension notification criteria do not include a criterion (e.g., any criterion) based on a pressure measurement.

In response to receiving the first sensor data from the one or more sensors (704) (e.g., 618, 620, and/or 622) and in accordance with a determination that the first sensor data from the one or more sensors (e.g., 618, 620, and/or 622) does not satisfy the set of hypertension notification criteria (e.g., the computer system uses the data from the one or more sensors to make an assessment as to whether the user associated with the computer system is at risk for chronic hypertension (e.g., the user associated with the computer system does not have consistently high blood pressure); the computer system uses the data from the one or more sensors to generate a risk score indicative of chronic hypertension and the risk score does not exceed the threshold value), computer system forgoes displaying the notification (e.g., 628) of the hypertension event (e.g., a notification is not displayed by the computer system and the external device because the data from the one or more sensors indicates that the user associated with the computer system is not at risk for chronic hypertension).

After (e.g., in response to; within a predetermined time of) computer system displays the notification (e.g., 628) of the hypertension event, computer system displays, via a display generation component, a notification (e.g., 866) prompting the user to initiate a process to receive a second set of one or more inputs to select a third time range and for displaying a third user interface (e.g., 800) (e.g., that includes one or more features of the first user interface) that includes a fourth plurality of measurement indicators (e.g., 836 and/or 840) that includes a number of measurement indicators selected based on the third time range.

Automatically generating a prompting notification after displaying a notification of a hypertension event surfaces to the user a relevant function, without requiring further user input and therefore improves and sustains user-system interactions. Performing an operation automatically and sustaining the user-system interaction enhances the operability of the computer system and makes the user-system interface more efficient (e.g., by helping the user to provide proper inputs and reducing user mistakes when operating/interacting with the computer system) which, additionally, reduces power usage and improves battery life of the system by enabling the user to use the computer system more quickly and efficiently.

Note that details of the processes described above with respect to method 900 (e.g., FIG. 9) are also applicable in an analogous manner to the methods described above.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to provide users with an indication of one or more health parameters and/or one or more health events. The present disclosure contemplates that in some instances, this gathered data may include personal information data that uniquely identifies or can be used to contact or locate a specific person. Such personal information data can include demographic data, location-based data, telephone numbers, email addresses, twitter IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to facilitate a third party assess a health of the user. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of hypertension notifications, the present technology can be configured to allow users to select to “opt in” or “opt out” of participation in the collection of personal information data during registration for services or anytime thereafter. In another example, users can select not to provide blood pressure data. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, captured sensor data and/or received blood pressure data can be maintained entirely on a user's electronic device and access to the data corresponding to the captures sensor data and/or received blood pressure data by another device (e.g., a server) can be prohibited without the user's express consent.

What is claimed is:

1. A wearable computer system, comprising:
one or more input devices;
one or more output devices;
one or more sensors, wherein the one or more sensors include a photoplethysmogram sensor and an electrocardiogram sensor;
one or more processors; and
memory storing one or more programs configured to be executed by the one or more processors, the one or more programs including instructions for:
receiving, via the one or more input devices, user input corresponding to a request to transition an activation state of hypertension notifications from a first state to a second state;
in response to receiving the user input corresponding to the request to transition the activation state of the hypertension notifications from the first state to the second state:
in accordance with a determination that the second state is an active state and that an age of a user of the wearable computer system is above a threshold age, enabling hypertension notifications; and
in accordance with a determination that the second state is the active state and that the age of the user of the wearable computer system is below the threshold age, forgoing enabling hypertension notifications;
after receiving the user input corresponding to the request to transition the activation state of the hypertension notifications from the first state to the second, receiving, over a predetermined period of time, first data associated with the user of the wearable computer system from the one or more sensors, wherein the first data from the one or more sensors does not include a direct measurement of pressure, and wherein the first data from the one or more sensors includes data selected from the group consisting of: blood volume data, movement data, heart rate data, and a combination thereof;
in response to receiving the first data from the one or more sensors and in response to the predetermined period of time elapsing:
in accordance with a determination that the second state is the active state:
in accordance with a determination that the first data from the one or more sensors satisfies a set of hypertension notification criteria, generating, via the one or more output devices, a notification indicative of a hypertension event associated with the user of the wearable computer system, wherein the notification indicative of the hypertension event indicates that the user of the computer system is at risk for chronic hypertension based on the first data associated with the user of the wearable computer system; and
in accordance with a determination that the first data from the one or more sensors does not satisfy the set of hypertension notification criteria, forgoing generating the notification indicative of the hypertension event;
in accordance with a determination that the second state is an inactive state, forgoing generating the notification indicative of the hypertension event irrespective of whether or not the first data from the one or more sensors satisfies the set of hypertension criteria; and
after generating the notification indicative of the hypertension event associated with the user of the wearable computer system in accordance with the determination that the second state is the active state and that the first data from the one or more sensors satisfies the set of hypertension notification criteria, prompting the user of the wearable computer system to initiate a data collection log for a predefined time period.

2. The wearable computer system of claim 1, wherein the predetermined period of time is selected from the group consisting of: a period of greater than 1 hour; a period of greater than 1 day; and a period of greater than 1 week.

3. The wearable computer system of claim 1, wherein:
the first data associated with the user of the wearable computer system includes a plurality of subsets of data that includes:
a first subset of data that is received over a second period of time; and
a second subset of data that is received starting after a third period of time has elapsed after receiving the first subset of data, wherein the third period of time is greater than the second period of time.

4. The wearable computer system of claim 1, wherein the set of hypertension notification criteria includes a criterion that is satisfied when a risk score exceeds a threshold value, wherein the risk score is generated based on the first data associated with the user of the wearable computer system.

5. The wearable computer system of claim 1, wherein the first data associated with the user of the wearable computer system does not include data from a vibration sensor.

6. The wearable computer system of claim 1, wherein the wearable computer system is not in communication with a component that actively applies mechanical pressure to the user of the wearable computer system, and wherein the one or more sensors do not include a sensor that measures data within a fourth period of time after active, mechanical pressure is applied to the user of the wearable computer system.

7. The wearable computer system of claim 1, wherein the one or more programs further include instructions for:
after receiving the first data associated with the user of the wearable computer system from the one or more sensors over the predetermined period of time, receiving second data associated with the user of the wearable computer system from the one or more sensors, wherein the second data is received over a second predetermined period of time that does not overlap the predetermined period of time;
in response to receiving the second data from the one or more sensors:
in accordance with a determination that the second data from the one or more sensors satisfies the set of hypertension notification criteria, generating, via the one or more output devices, a second notification indicative of a hypertension event associated with the user of the wearable computer system.

8. The wearable computer system of claim 1, wherein the one or more output devices includes a display generation component, and wherein the one or more programs further include instructions for:
receiving a first set of one or more inputs, via the one or more input devices, corresponding to the notification;
in response to the first set of one or more inputs, displaying, via the display generation component, a first user interface that includes first information corresponding to the first data.

9. The wearable computer system of claim 1, wherein the one or more output devices includes a display generation component, and wherein the one or more programs further include instructions for:
after generating the notification indicative of a hypertension event, displaying, via the display generation component, a second user interface that includes information corresponding to one or more notifications indicative of hypertension events, including the notification indicative of a hypertension event.

10. The wearable computer system of claim 1, wherein the notification indicative of a hypertension event does not include a numerical indication of a blood pressure value of a user of the wearable computer system.

11. The wearable computer system of claim 1, wherein:
the first data associated with the user of the wearable computer system includes a second plurality of subsets of data that includes:
a third subset of data that is received at a first time, wherein the third subset of data is not received in response to user input; and
a fourth subset of data that is received a second predetermined period of time after receiving the third subset of data.

12. The wearable computer system of claim 1, wherein the wearable computer system determines that the first data from the one or more sensors satisfies the set of hypertension notification criteria based on trends corresponding to chronic hypertension identified using a machine learning algorithm.

13. A non-transitory computer-readable storage medium storing one or more programs configured to be executed by one or more processors of a wearable computer system that is in communication with one or more input devices, one or more output devices, and one or more sensors, wherein the one or more sensors include a photoplethysmogram sensor and an electrocardiogram sensor, the one or more programs including instructions for:
receiving, via the one or more input devices, user input corresponding to a request to transition an activation state of hypertension notifications from a first state to a second state;
in response to receiving the user input corresponding to the request to transition the activation state of the hypertension notifications from the first state to the second state:
in accordance with a determination that the second state is an active state and that an age of a user of the wearable computer system is above a threshold age, enabling hypertension notifications; and
in accordance with a determination that the second state is the active state and that the age of the user of the wearable computer system is below the threshold age, forgoing enabling hypertension notifications;
after receiving the user input corresponding to the request to transition the activation state of the hypertension notifications from the first state to the second, receiving, over a predetermined period of time, first data associated with the user of the wearable computer system from the one or more sensors, wherein the first data from the one or more sensors does not include a direct measurement of pressure, and wherein the first data from the one or more sensors includes data selected from the group consisting of: blood volume data, movement data, heart rate data, and a combination thereof;
in response to receiving the first data from the one or more sensors and in response to the predetermined period of time elapsing:
in accordance with a determination that the second state is the active state:
in accordance with a determination that the first data from the one or more sensors satisfies a set of hypertension notification criteria, generating, via the one or more output devices, a notification indicative of a hypertension event associated with the user of the wearable computer system, wherein the notification indicative of the hypertension event indicates that the user of the computer system is at risk for chronic hypertension based on the first data associated with the user of the wearable computer system; and
in accordance with a determination that the first data from the one or more sensors does not satisfy the set of hypertension notification criteria, forgoing generating the notification indicative of the hypertension event;
in accordance with a determination that the second state is an inactive state, forgoing generating the notification indicative of the hypertension event irrespective of whether or not the first data from the one or more sensors satisfies the set of hypertension criteria; and
after generating the notification indicative of the hypertension event associated with the user of the wearable computer system in accordance with the determination that the second state is the active state and that the first data from the one or more sensors satisfies the set of hypertension notification criteria, prompting the user of the wearable computer system to initiate a data collection log for a predefined time period.

14. A method, comprising:
at a wearable computer system that is in communication with one or more input devices, one or more output devices, and one or more sensors, wherein the one or more sensors include a photoplethysmogram sensor and an electrocardiogram sensor:
receiving, via the one or more input devices, user input corresponding to a request to transition an activation state of hypertension notifications from a first state to a second state;
in response to receiving the user input corresponding to the request to transition the activation state of the hypertension notifications from the first state to the second state:
in accordance with a determination that the second state is an active state and that an age of a user of the wearable computer system is above a threshold age, enabling hypertension notifications; and
in accordance with a determination that the second state is the active state and that the age of the user of the wearable computer system is below the threshold age, forgoing enabling hypertension notifications;
after receiving the user input corresponding to the request to transition the activation state of the hypertension notifications from the first state to the second, receiving, over a predetermined period of time, first data associated with the user of the wearable computer system from the one or more sensors, wherein the first data from the one or more sensors does not include a direct measurement of pressure, and wherein the first data from the one or more sensors includes data selected from the group consisting of: blood volume data, movement data, heart rate data, and a combination thereof;
in response to receiving the first data from the one or more sensors and in response to the predetermined period of time elapsing:
in accordance with a determination that the second state is the active state:
in accordance with a determination that the first data from the one or more sensors satisfies a set of hypertension notification criteria, generating, via the one or more output devices, a notification indicative of a hypertension event associated with the user of the wearable computer system, wherein the notification indicative of the hypertension event indicates that the user of the computer system is at risk for chronic hypertension based on the first data associated with the user of the wearable computer system; and
in accordance with a determination that the first data from the one or more sensors does not satisfy the set of hypertension notification criteria, forgoing generating the notification indicative of the hypertension event;
in accordance with a determination that the second state is an inactive state, forgoing generating the notification indicative of the hypertension event irrespective of whether or not the first data from the one or more sensors satisfies the set of hypertension criteria; and
after generating the notification indicative of the hypertension event associated with the user of the wearable computer system in accordance with the determination that the second state is the active state and that the first data from the one or more sensors satisfies the set of hypertension notification criteria, prompting the user of the wearable computer system to initiate a data collection log for a predefined time period.
